(19) United States
(12) Patent Application Publication
(10) Pub. No.: US 2004/0136959 A1
(43) Pub. Date: Jul. 15, 2004

(54) Sensitization of cancer cells to immunoconjugate-induced cell death by transfection with il -13 receptor alpha chain

(76) Inventor: Raj K. Puri, Potomac, MD (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/250,998

(22) PCT Filed: Aug. 15, 2001
(86) PCT No.: PCT/US01/25663

(51) Int. Cl. A61K 48/00

(52) U.S. Cl. 424/93.2; 514/44

(57) ABSTRACT

The invention relates to the discovery that cancer cells that have no or low expression of the IL-13 receptor (“IL-13R”) can bind IL-13R-targeted immunoconjugates, such as immunotoxins, by transfection with the IL-13Rα2 chain alone. Transfecting cells with just the IL-13Rα2 chain is easier than transfection with an intact receptor. For some cancers, transfection with the IL-13Rα2 chain alone inhibits tumor growth. Those cancers that are not inhibited by the presence of the IL-13Rα2 chain alone, and which do not express the IL-13R or express it only at low levels can be rendered sensitive to IL-13R-targeted immunoconjugates by transfection of the IL-13α2 chain and can be inhibited by the use of immunoconjugates, such as immunotoxins, targeted to the IL-13R. Nucleic acids encoding the IL-13Rα2 chain or vectors containing such nucleic acids can be used for the manufacture of medicaments to introduce the IL-13Rα2 chain into cancer cells and thereby either inhibit their growth (for cells inhibited by the presence of the IL-13Rα2 chain) or to sensitize them to IL-13R-targeted immunoconjugates, or both.

### CROSS-REFERENCES TO RELATED APPLICATIONS

This application claims priority from U.S. Provisional Patent Application No. 60/229,842, filed Aug. 31, 2000, the contents of which are hereby incorporated by reference for all purposes.

### STATEMENT AS TO RIGHTS TO INVENTIONS MADE UNDER FEDERALLY SPONSORED RESEARCH AND DEVELOPMENT

Not applicable.

### REFERENCE TO A “SEQUENCE LISTING,” A TABLE, OR A COMPUTER PROGRAM LISTING APPENDIX SUBMITTED ON A COMPACT DISC

Not applicable.

### FIELD OF THE INVENTION

This invention relates to transfecting cancer cells with the IL-13 receptor α2 chain to sensitize them to agents delivered by IL-13 receptor targeted immunoconjugates.

### BACKGROUND OF THE INVENTION

Targeting of cell surface proteins on cancer cells is a modern approach for cancer therapy (Vitetta, E. S. et al., Science 238, 1098-1101 (1988); Pastan, I. et al., Science 254, 1173-1177 (1991); Uckun, F. M. et al., Br. J. Haematol. 85,435-438 (1993); Murphy, J. R. et al., Cancer Biol. 6, 259-267 (1995); Youle, R. J., Cancer Biol. 7, 65-70 (1996); Puri, R. K. et al., Toxicol. Pathol. 27, 53-57 (1999)). Targeted cytotoxins are 5-10 times more potent on cancer cells than chemotherapy and provide specificity without producing undesirable side effects (Frankel, A. E. et al., Cancer Res. 56, 926-932 (1996); Rand, R. W. et al., Clin. Cancer Res. 6, 2157-2165 (2000)). To generate a targeted agent, identification of unique cancer cell-associated receptors or antigens is important. Plasma membrane receptors for the helper T cell type 2 (TH2)-derived cytokine interleukin 13 (IL-13) have been identified on a variety of human solid cancer cells (Debinski, W. et al., J. Biol. Chem. 270, 16775-16180 (1995); Debinski, W. et al., Clin. Cancer Res. 1, 1253-1258 (1995); Obiri, N. I. et al., J. Biol. Chem. 270, 8797-8804 (1995); Puri, R. K. et al., Blood 87, 4333-4339 (1996); Husain, S. R. et al., Clin. Cancer Res. 3, 151-156 (1997); Maini, A. et al., J. Urol. 158, 948-953 (1997); Murata, T. et al., Cell. Immunol. 175, 33-40 (1997); Murata, T. et al., Int. J. Cancer 70, 230-240 (1997); Husain, S. R. et al., Blood 95, 3506-3513 (2000); Josh B. H. et al., Cancer Res. 60, 1168-1172 (2000)). Interleukin 13 plays a major role in inflammatory diseases (Wills-Karp, M. et al., Science 282, 2258-2261 (1998)) and may play a prominent role in cancer as receptors for this cytokine are overexpressed on some cancer cells.

Unlike receptors for related cytokine IL-4, the receptors for IL-13 have not been well characterized. The structure of the IL-13 receptor (“IL-13R”) has been studied in various cell types (Obiri, N. I. et al., J. Biol. Chem. 270, 8797-8804 (1995); Obiri, N. I. et al., J. Immunol. 158, 756-764 (1997); Obiri, N. I. et al., J. Biol. Chem. 272, 20251-20258 (1997); Murata, T. et al., Cell. Immunol. 175, 33-40 (1997); Murata, T. et al., Int. J. Cancer 70, 230-240 (1997); Murata, T. et al., Int. Immunol. 10. 1103-1110 (1998); Murata, T. et al., Int. J. Mol. Med. 1, 551-557 (1998)). It has been reported that IL-13 binds to two isoforms of 65-kDa proteins in human renal cell carcinoma cells, and that one of these proteins also binds IL-4 (Obiri, N. I. et al., J. Biol. Chem. 270, 8797-8804 (1995)). On the basis of the binding characteristics, cross-linking, and displacement of radiolabeled IL-4 and IL-13 in various cell types, it has been hypothesized that like the IL-4R system, IL-13R may also exist as three different types (Murata, T. et al., Cell. Immunol. 175, 3340 (1997); Murata, T. et al., Int. J. Cancer 70, 230-240 (1997a); Murata, T. et al., Int. Immunol. 10. 1103-1110 (1998a); Murata, T. et al., Int. J. Mol. Med. 1, 551-557 (1998b); Obiri, N. I. et al., J. Immunol. 158, 756-764 (1997a); Obiri, N. I. et al., J. Biol. Chem. 272, 20251-20258 (1997b)). Two different chains (IL-13Rα′ and IL-13Rα) of the IL-13R system have been cloned, and correspond to two of the 65-kDa isoforms originally proposed (Obiri, N. I. et al., J. Biol. Chem. 270, 8797-8804 (1995)). The murine and human IL-13Rα′ chain (also known as IL-13Rα₁) was cloned first (Aman, M. J. et al., J. Biol. Chem. 271, 29265-29270 (1996); Hilton, D. J. et al., Proc. Natl. Acad. Sci. U.S.A. 93, 497-501 (1996)). This chain binds IL-13 at low level but when coupled with the IL-4Rβ chain (also known as IL-4Rα) binds IL-13 and mediates IL-13-induced signaling (Miloux, B. et al., FEBS Lett. 401, 163-166 (1997)). The second chain of IL-13R, termed IL-13Rα (now also known as IL-13Rα₂ or IL-13Rα2), was cloned from a human renal cell carcinoma cell line (Caki-1). This chain has 50% homology to IL-5R at the DNA level has a short intracellular domain, and binds IL-13 with high affinity (Caput, D. et al., J. Biol. Chem. 271, 16921-16926 (1996)).

The IL-13Rα2 chain plays an important role in IL-13 binding and internalization in the IL-13R system. Although IL-13R is expressed on many cancer cell lines, some cell lines do not express, or express only a low level of; the α2 chain. Because of low-level expression of IL-13Rα2 chain, these cells show no, or only low, sensitivity to an IL-13R-targeted cytotoxin. IL13-PE38QQR, which is composed of IL-13 and a mutated form of a Pseudomonas exotoxin (Debinski, W. et al., J. Biol. Chem. 270, 16775-16180 (1995a); Debinski, W. et al., Clin. Cancer Res. 1, 1253-1258 (1995b); Puzi, R. K. et al., Blood 87, 4333-4339 (1996a); Husain, S. R. et al., Clin. Cancer Res. 3, 151-156 (1997); Maini, A. et al., J. Urol. 158, 948-953 (1997); Husain, S. R. et al., Blood 95, 3506-3513 (2000)).

### BRIEF SUMMARY OF THE INVENTION

This invention provides the ability to sensitize cancer cells to IL-13R-targeted immunoconjugates. In one important group of embodiments, the invention provides the use of a vector encoding a polypeptide with at least 70% identity to an amino acid of a IL-13 receptor α2 chain (SEQ ID NO:1) to manufacture a medicament for sensitizing a cancer cell to an immunotoxin binding to an IL-13Rα2 chain, provided that said encoded polypeptide can bind IL-13. In preferred embodiments, the encoded polypeptide has at least 80% identity to an IL-13 receptor α2 chain (SEQ ID NO:1), and in more preferred embodiments, the encoded polypeptide has at least 90% identity to an IL-13 receptor α2 chain (SEQ ID NO:1). In the most preferred embodiment, the encoded polypeptide has the sequence of IL-13 receptor α2 chain (SEQ ID NO:1). In preferred embodiments, the cancer cell is a cell from a cancer selected from the group consisting of: a brain cancer, a head and neck cancer, a breast cancer, a liver cancer, a lung cancer, a mesothelioma, a pancreatic cancer, a colon cancer, a gastric cancer, an ovarian cancer, a renal cancer, a bladder cancer, a prostate cancer, a testicular cancer, a skin cancer, a cervical cancer, a uterine cancer, and a sarcoma. In one preferred embodiment, the head and neck cancer is a squamous cell carcinoma.

In a further group of embodiments, the invention provides the use of a vector encoding a polypeptide with at least 70% identity to an amino acid of a IL-13 receptor α2 chain (SEQ ID NO:1) for the manufacture of a medicament for inhibiting the growth of a cancer cell, provided that said encoded polypeptide can bind IL-13. In preferred embodiments, the encoded polypeptide has at least 80% identity to an IL-13 receptor α2 chain (SEQ ID NO:1). In more preferred embodiments, the encoded polypeptide has at least 90% identity to an IL-13 receptor α2 chain (SEQ ID NO:1). In the most preferred embodiment, the encoded polypeptide has the sequence of IL-13 receptor α2 chain (SEQ ID NO:1). In some embodiments, the cancer cell is a cell from a cancer selected from the group consisting of a breast cancer and a pancreatic cancer.

The invention further provides compositions comprising a nucleic acid encoding a polypeptide with at least 70% identity to an IL-13 receptor α2 chain (SEQ ID NO:1) operably linked to a promoter, and a pharmaceutically acceptable carrier, provided that said encoded polypeptide can bind IL-13. In preferred embodiments, the compositions comprise a polypeptide with at least 80% identity to an IL-13 receptor α2 chain (SEQ ID NO:1). In more preferred embodiments, the composition comprises aa polypeptide has at least 90% identiiy to an IL-13 receptor α2 chain (SEQ ID NO:1). In the most preferred embodiment, the polypeptide has the sequence of an IL-13 receptor α2 chain (SEQ ID NO:1).

In yet another group of embodiments, the invention provides methods for inhibiting the growth of a cancer tumor, said method comprising transfecting at least some cells of said tumor with a nucleic acid sequence encoding a polypeptide with at least 70% identity to an IL-13Rα2 chain (SEQ ID NO:1), provided said encoded polypeptide can bind IL-13. In preferred embodiments, the encoded polypeptide has at least 80% identity to an IL-13Rα2 chain (SEQ ID NO:1). In more preferred embodiments, the encoded polypeptide has at least 90% identity to an IL-13Rα2 chain (SEQ ID NO:1). In the most preferred embodiments, the encoded polypeptide has the sequence of an IL-13Rα2 chain (SEQ ID NO:1). In some embodiments, the cancer tumor is selected from the group consisting of a pancreatic cancer and a breast cancer.

In a further group of embodiments, the invention provides methods for sensitizing a cancer cell to an effector molecule, the method comprising transfecting said cell with a nucleic acid sequence encoding a polypeptide with at least 70% identity to an IL-13Rα2 chain (SEQ ID NO:1), provided said encoded polypeptide can bind IL-13. In preferred embodiments, the encoded protein has at least 85% identity to an IL-13Rα2 chain (SEQ ID NO:1), provided said encoded polypeptide can bind IL-13. In more preferred embodiments, the encoded polypeptide has the sequence of an IL-13Rα2 chain (SEQ ID NO:1). In some embodiments, the methods further comprise contacting the cell with an immunoconjugate comprising a targeting moiety and an effector moiety, wherein said targeting moiety is a ligand for the IL-13Rα2 chain (SEQ ID NO:1). In preferred embodiments, the ligand is selected from the group consisting of IL-13, a mutated IL-13, which mutated IL-13 retains the ability to bind to an IL-13Rα2 chain (SEQ ID NO:1), a circularly permuted IL-13 (“cpIL-13”), and an antibody that specifically binds to an IL-13Rα2 chain (SEQ ID NO:1). In preferred embodiments, the ligand is IL-13, or a fragment of IL-13, which fragment of IL-13 retains the ability to bind to an IL-13Rα2 chain (SEQ ID NO:1), a cpIL-13, which cpIL-13 retains the ability to bind to an IL-13Rα2 chain (SEQ ID NO:1), a mutated IL-13, which mutated IL-13 retains the ability to bind to an IL-13Rα2 chain (SEQ ID NO:1), or an anti-IL-13Rα2 chain antibody. In some embodiments, the anti-IL-13Rα2 chain antibody is a single chain Fv or a disulfide-stabilized Fv. The cancer cell can be, for example, a cell from a cancer selected from the group consisting of: a brain cancer, a head and neck cancer, a breast cancer, a liver cancer, a lung cancer, a mesothelioma, a colon cancer, a gastric cancer, an ovarian cancer, a renal cancer, a bladder cancer, a prostate cancer, a pancreatic cancer, a testicular cancer, a skin cancer, a cervical cancer, a uterine cancer, and a sarcoma. In some embodiments, the head and neck cancer is a squamous cell carcinoma. The effector moiety can be selected from the group consisting of cytotoxin, a radionuclide, a radioisotope, a drug, and a liposome, wherein the liposome contains a cytotoxin, a radionuclide, or a drug. In some embodiments, the cytotoxin is selected from the group consisting of ricin A, abrin, ribotoxin, ribonuclease, saporin, calicheamycin, diphtheria toxin or a subunit thereof, Pseudomonas exotoxin, a cytotoxic portion thereof a mutated Pseudomonas exotoxin, a cytotoxic portion thereof, and botulinum toxins A through F.

In preferred embodiments, the cytotoxin is a Pseudomonas exotoxin or cytotoxic fragment thereof, or a mutated Pseudomonas exotoxin or a cytotoxic fragment thereof. In particularly preferred embodiments, the Pseudomonas exotoxin is selected from the group consisting of PE35, PE38, PE38KDEL, PE40, PE4E, and PE38QQR.

### BRIEF DESCRIPTION OF THE DRAWINGS

FIG. 1. Binding of ¹²⁵I-IL-13 to cancer cells transfected with IL-13Rα2 chain.

The four lettered graphs show the results of various cell types incubated at 4° C. for 2 hr with 200 pM ¹²⁵I-labeled IL-13 with or without 40 nM unlabeled IL-4 or IL-13. The cell types were: FIG. 1A: T98G, FIG. 1B: A253, FIG. 1C: Caki-1, FIG. 1D: PANC-1. For each experiment, cells (5×10⁵) were transfected either with the vector alone or with the vector encoding the IL-13Rα2 chain. Data represent the mean of duplicate determinations; bars represent the SD.

FIG. 2. Cytotoxicity of IL-13 toxin to cancer cells transfected with vector alone (control) or IL-13R α2 chain.

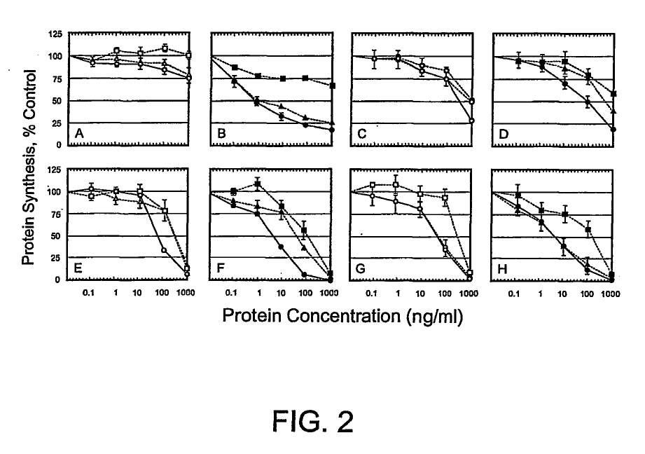

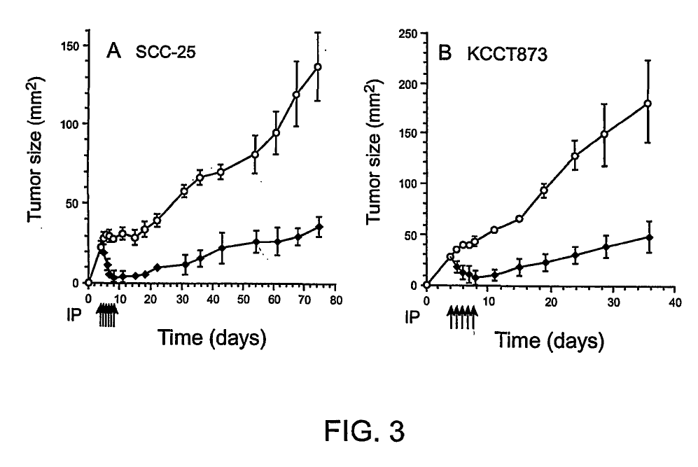

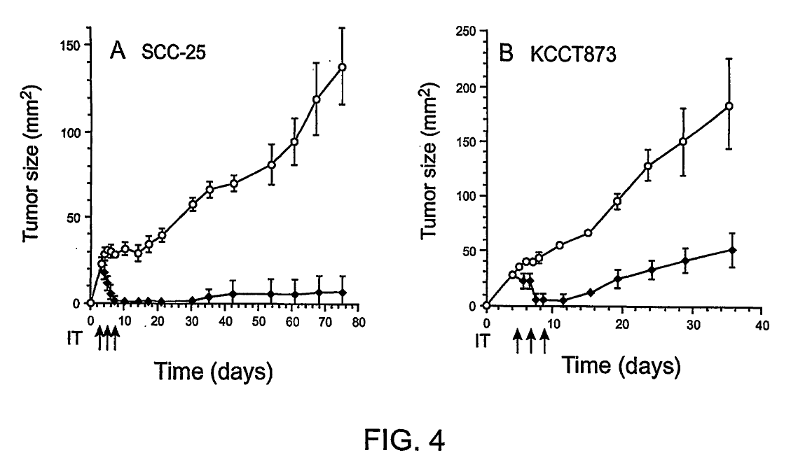

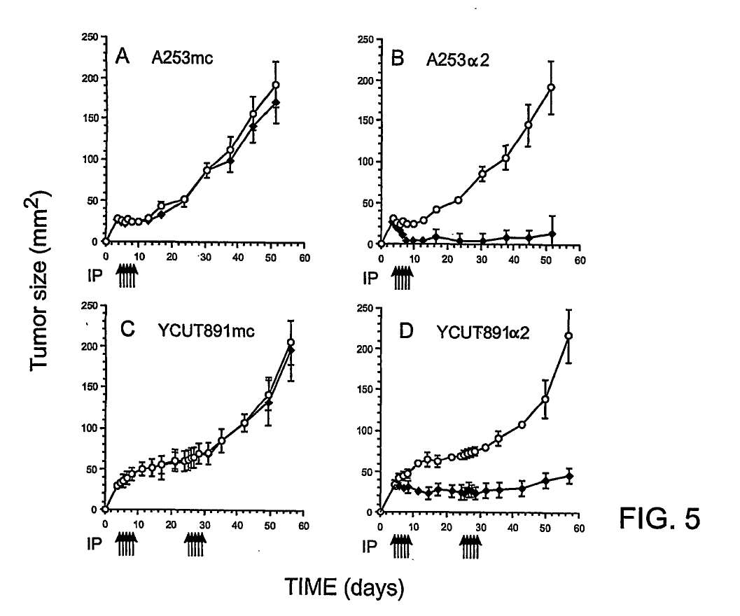

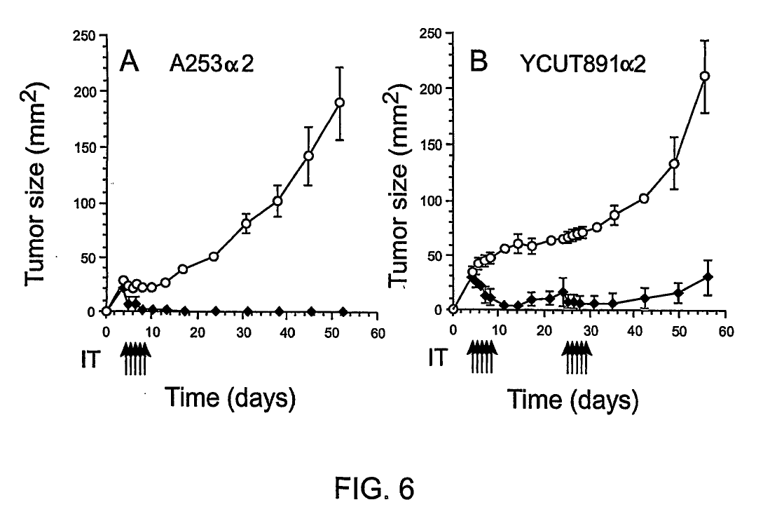

Each pair of lettered graphs in FIG. 2 shows the results for a particular cell line transfected with the vector alone, as a control, or with the IL-13R α2 chain FIGS. 2A and B show the results of transfecting T98G cells with the vector (FIG. 2A) or with IL-13R α2 (FIG. 2B). FIGS. 2C and D show the results of transfecting Caki-1 cells with the vector (FIG. 2C) or with IL-13R α2 (FIG. 2D). FIGS. 2E and F show the results of transfecting A253 cells with the vector (FIG. 2E) or with IL-13R α2 (FIG. 2F). FIGS. 2G and H show the results of transfecting PANC-1 cells with the vector (FIG. 2G) or with IL-13R α2 (FIG. 2H) (PANC-1 cells are a pancreatic cancer cell line). For all the figures in FIG. 2, the cells were cultured with various concentrations of IL13-PE38QQR (0-1000 ng/ml) with or without IL-4 or IL-13 (2 μg/ml). The results are represented as means ±SD of quadruplicate determinations, and the assay was repeated several times. The concentration of IL13-PE38QQR at which 50% inhibition of protein synthesis (IC₅₀) occurred was calculated. For the figures showing the results in cell transfected with the control (vector alone), the following symbols are used to indicate the presence of the cytokines and immunoconjugates to which the cells were exposed in the studies: IL13-PE38QQR: (◯); IL13-PE38QQR+IL-4: (Δ); IL13-PE38QQR+IL-13: (¤). For the figures showing the cells transfected with IL13Rα2 chain: IL13-PE38QQR: (●);IL13-PE38QQR+IL4: (▴); IL13-PE38QQR+IL-13: (▪).

FIG. 3 Regression of IL-13Rα2 chain-positive SCCHN tumors by intraperitoneal administration of IL-13 toxin. FIG. 3A: Nude mice were implanted subcutaneously with 5×10⁶ SCC-25 cells on day 0. The animals then received twice a day injections with IL-13 toxin (50 μg/kg) for 5 days from day 4 to 8 (♦). The control mice were injected with excipient only (◯). Each group had 5 animals. The arrows indicate the day of injections; bars, SD. FIG. 3B: Nude mice implanted subcutaneously with 5×10⁶ KCCT873 cells on day 0. All other parameters were the same as described for FIG. 3A.

FIG. 4 Regression of IL-13Rα2 chain-positive SCCHN tumors by intratumoral injections of IL-13 toxin. FIG. 4A: Nude mice with established SCC-25 tumore. FIG. 4B: Nude mice implanted with KCCT873 tumors. Mice in both Figures received 250 μg/kg of IL-13 toxin (♦) or excipient only (◯) on days 4, 6, and 8. Control group had 5 mice and treated group had 4 mice. The injected volume was 30 μl in each tumor. The arrows indicate the day of injections; bars, SD.

FIG. 5 Regression of IL-13Rα2 chain-transfected SCCHN tumors by intraperitoneal administration of IL-13 toxin. FIGS. 5A and 5C: Nude mice were implanted subcutaneously with 5×10⁶ vector only transfected cells A253mc FIG. 5A) and YCUT891mc (FIG. 5C) on day 0. FIGS. 5B and D: Nude mice were implanted subcutaneously with 5×10⁶ IL-13Rα2 chain transfected cells A253α2 (FIG. 5B) or YCUT891α2 (FIG. 5D) on day 0. All figures in FIGS. 5A-D: The animals then received twice a day injections with IL-13 toxin (50 μg/kg) (♦) or excipient only (◯) for 5 days as the arrows indicated. YCUT891mc and YCUT891α2 tumor bearing mice (FIGS. 5C and 5D, respectively) received a second course of injections on days 25 to 29 after implantation with same dose of IL-13 toxin as the first course. Each group of mice had 5 animals; bars, SD.

FIG. 6. Complete regression of IL-13Rα2 chain-transfected SCCHN tumors by intratumoral injections of IL-13 toxin. Nude mice with established A253α2 tumors (FIG. 6A) or YCUT891α2 tumors (FIG. 6B) received 250 μg/kg of IL-13 toxin (♦) or excipient only (◯) as the arrows indicated. YCUT891α2 tumor bearing mice received a second course of injection on day 25, 27, and 29 of implantation with same dose of IL-13 toxin as the first course. The injected volume was 30 μl in each tumor and each group had 5 animals; bars, SD.

### DETAILED DESCRIPTION

Introduction

Cells of a number of cancers overexpress the receptor for interleukin-13 hereafter, “IL-13,” the receptor for IL-13 is abbreviated as “IL-13R”). Recent studies have-shown that growth of these IL-13R-overexpressing cancers can be inhibited by contacting the cancers with chimeric molecules formed by fusing or conjugating a targeting molecule (which becomes a targeting “moiety” once fused or conjugated), such as IL-13, a circularly permuted (“cp”) form of IL-13, or an anti-IL-13R antibody, with an effector molecule (which can be referred to as a “effector moiety” or “effector molecule” once fused or conjugated), such as a radioisotope, drug, or cytotoxin. Such chimeric molecules are sometimes referred to as immunotoxins. Typically, the targeting moiety binds the immunotoxin to the IL-13R, permitting internalization of the immunotoxin, and the subsequent death of the cell. For convenience, these IL-13 receptor-targeted chimeric conjugates will be referred to herein as “IL-13R-targeted conjugates,” “IL-13R immunotoxins,” or “IL-13R chimeric toxins.”

It is known that the IL-13 receptor is heteromeric, and is composed of several distinct chains. Moreover, as noted in the Background, the IL-13R may exist in three different forms. It has now been noted that the α2 chain of the IL-13 receptor is either not expressed or is expressed only at low levels in cancers that show no or low sensitivity to IL-13-targeted conjugates. For example, as reported in the Examples herein, only 20% of 17 cell lines of squamous cell carcinomas of the head and neck (“SCCHN”) studied were found to express high levels of IL-13R.

Surprisingly, although the IL-13 receptor comprises several chains and appears to exist in several forms, it has now been discovered that cancer cells that do not express the IL-13R, or express it at only low levels, can be made sensitive to IL-13R-targeted conjugate by transfection with just the IL-13Rα2 chain. Surprisingly, transfection with this single chain renders cells of a number of cancers otherwise insensitive to IL-13R-targeted conjugates, such as immunotoxins, up to 1000 times more sensitive to such immunoconjugates than are non-transfected controls.

Even more surprisingly, it has now also been discovered that the transfection of at least some cells of a tumor with the IL-13Rα2 chain not only renders the transfected cells susceptible to inhibition or killing by contacting them with an IL-13-targeted chimeric toxin, but also results in the inhibition or death of other cells in the tumor whether or not they themselves were transfected with the IL-13R α2 chain. In vivo studies in which established tumors of different types of cancers were transfected with the IL-13Rα2 chain and then contacted with an exemplary IL-13R-targeted immunoconjugate by either systemic administration or by intratumoral administration, demonstrated significant inhibition or even complete regression of the tumors, despite the fact that not every cell of the tumor was transfected with the IL-13Rα2 chain.

Without wishing to be bound by theory, it is believed that transfection of at least some of cells of a tumor with the IL-13Rα2 chain may cause either the cells so transfected, or other cells of the tumor, to secrete a cytokine or other factor that attracts neutrophils, macrophages, or other lymphocytes to the tumor, and that these cells are then activated to kill tumor cells whether or not the particular tumor cells killed were transfected with the chain.

Additionally, in vivo studies with two different cancers indicate that the IL-13Rα2 chain itself inhibits the growth of some cancers. In these studies, using widely used pancreatic cancer and breast cancer cells, cells transfected with the IL-13Rα2 chain did not grow when implanted in a nude mouse model, while like cells transfected with just the vector grew robustly into large tumors. Thus, transfecting the cells of some cancers with the IL-13Rα2 chain itself inhibits the growth of some cancers. It should be noted that the effects discussed in this paragraph and in the preceding paragraph were noted in a nude mouse model; they are expected to be even more robust in mammals with an intact cellular immune response. Additional cancers susceptible by inhibition by the presence of the IL-13Rα2 chain can be determined simply by transfecting cells of the cancer of interest and determining whether the cells can grow into a tumor mass in a nude mouse model compared to cells transfected only with the vector (known as mock-transfection).

The discoveries of the invention provide a number of advantages. Importantly, the invention extends the cancers that can be inhibited by IL-13R-targeted immunoconjugates beyond the limited range of cancers that naturally overexpress the IL-13R Further, the discovery that cancers can be rendered susceptible to IL-13R-targeted immunoconjugates by transfection with a nucleic acid encoding a single chain of the IL-13R rather than one that encodes the entire receptor, with its multiple chains, makes it much easier to use IL-13R-targeted approaches. Even if the multiple chains of the receptor can self assemble into a functional receptor, the transfection of a smaller nucleic acid encoding a single chain can be expected to be easier and to have a higher probability of success than transfection of one nucleic acid encoding several chains, or of several nucleic acids, each of which encodes a separate chain. The fact that;a smaller amount of nucleic acid is needed to transfect only a single chain of the receptor increases the number of vectors that can be used to transfect target cells, since all vectors have a limit to the amount of heterologous nucleic acid with which they can be loaded. Moreover, IL-13R-targeted conjugates, such as immunotoxins targeted to the IL-13R with IL-13 and IL-13 mutants with high binding affinity to the IL-13R, have been tested both preclinically and in clinical trials. E.g., Husain et al., Int. J. Cancer 92(2):168-75 (2001). These studies have indicated that IL-13R-targeted conjugates have little or no substantial toxicity to normal tissue.

The invention can be used in a number of ways. The tumors of many cancers localize in positions where they cannot be surgically resected because the surgery would cause unacceptable or fatal damage to an adjacent or surrounding vital organ. Cancers with localized tumors that can be transfected with the IL-13Rα2 chain include brain tumors, especially gliablastomas, head and neck cancers, especially squamous cell carcinomas, breast cancer, liver cancer, lung cancer, mesothelioma, colon cancer, gastric cancer, ovarian cancer, renal cancer, bladder cancer, prostate cancer, testicular cancer, skin cancers, especially melanoma, pancreatic cancer, cervical cancer, uterine cancer, and sarcomas. The present discovery permits a nucleic acid construct encoding the IL-13Rα2 chain to be introduced into cells of these cancers to increase their expression of the IL-13R α2 chain. Tumors whose growth is inhibited by the presence of the IL-13Rα2 chain will be inhibited by the presence of the chain. The effect can be enhanced in these cancers by contacting the tumor with an IL-13R-targeted chimeric toxin, which will then be bound and internalized by cells expressing IL-13Rα2 chain. The growth of these cells is then further inhibited by the cytotoxic action of the toxic moiety of the IL-13 conjugate. Tumors whose growth is not inhibited by the presence of the IL-13Rα2 chain itself can be contacted with an IL-13R-targeted conjugate, which will then be bound and internalized by cells expressing IL-13Rα2 chain. The growth of these cells is then inhibited by the action of the effector moiety of the IL-13 conjugate, such as a drug, radioisotope, or cytotoxin. Additionally, as described above, it has been discovered that even if only some of the cells of a IL-13R-targeted chimeric toxin are transfected, growth of some or all of the non-transfected cells is also inhibited. Based on the results in animal models, transfection of even a portion of cells of a tumor and subsequenct contacting of the tumor cells with an IL-13R-targeted conjugate, such as an immunotoxin, will result in inhibition of growth of the tumor and even in complete regression of the tumor.

Cells of the tumor can be transfected with nucleic acids encoding the IL-13Rα2 chain by any convenient means. Conveniently, the nucleic acid can be injected directly into cells of a tumor in so-called “needleless” “biolistic” devices or gene guns. The biolistic devices typically accelerate a particle, such as a gold particle coated with the nucleic acid of interest, directly into a tissue of interest. Gene guns typically accelerate a liquid containing a nucleic acid, or a dry formulation containing the nucleic acid, into the tissue by gas pressure. Such DNA can be in the form of a plasmid, can be so-called “naked” DNA, and can be circular or linearized. In some embodiments, the nucleic acids are stabilized with an excipient, often a carbohydrate such as trehalose, and may be lypophilized. If the tumor is on the skin or is otherwise rendered accessible (for example, by surgery which exposes the tumor), such devices can introduce the nucleic acids to be expressed directly into tumor cells and avoid concerns about uptake of the nucleic acid. Methods and devices for transfecting cells that may be utilized in the present invention are well known in the art and are taught in, for example, Felgner, et al., U.S. Pat. No. 5,703,055; Furth and Hennighausen, U.S. Pat. No. 5,998,382; Falo et al., WO 97/11605; Erdile et al., WO 99/26662; and Donnelly et al., WO 99/52463. See also, Sakaguchi et al., WO 96/12808;. Volkin et al., WO 97/40839; and Robinson et al., WO 95/20660. A variety of methods are known in the art for formulating microparticles suitable for needleless injection into a tissue. See, e.g., Osborne, WO 00/13668.

Conveniently, a nucleic acid encoding the IL-13Rα2 chain can also be transferred to cancer cells of interest by intratumoral injection. Depending on the location of the tumor, such injections can be made stereotactically, typically in conjunction with x rays of the affected area to assist the practitioner in placing the needle. Stereotactic injection is especially common in the case of brain and breast tumors. The practitioner can also be guided in making the injections by imaging technologies such as ultrasound which permit visualization of the needle and of the mass to be injected. Injections can also be made into tumors during arthroscopic or traditional surgery. The choice of how to access the tumor for transfection is within the expertise of the practitioner.

In some embodiments, the nucleic acids may be placed in a viral vector. Transfection by retroviral, adeno-associated virus, lentivirus, adenoviruses, and lentiviruses pseudotyped with vesiular stomatitis virus, canarypoxvirus, and chickenpox virus vectors, for example, has been taught in the art and can be employed in the practice of the invention. In some preferred embodiments, the nucleic acids are delivered in liposomes. Liposome encapsulation is preferred for needle injection since the liposomes tend to spread somewhat more than viral vectors in a tumor bed and therefore have the opportunity to transfect a somewhat larger number of cells of the tumor. U.S. Pat. No. 5,880,103 describes several methods of delivery of nucleic acids encoding peptides. The methods include liposomal delivery of the nucleic acids (or of the synthetic peptides themselves).

The IL-13R-targeted chimeric molecules, such as immunotoxins, can be administered either locally to the tumor by intratumoral injection or systemically. Conveniently, the chimeric molecules are administered intravenously. Typically, the chimeric molecules are administered in a pharmaceutically acceptable carrier. For i.v. administration, the immunoconjugates can be administered at a starting dosage of 0.5 microgram/kg, then 1 microgram/kg three times per week, and then escalated to 2 microgram/kg and then 3 microgram/kg per week, providing that the patient adequately tolerates the previous dosage. Intratumoral administration is started at 10 microgram/kg three times per week and the dosage is then doubled either until the patient shows adverse reaction to the administration or until the tumor shows complete regression. Traditionally, patients can tolerate higher doses administered by the IT route than by systemic (e.g., i.v.) routes. As noted above, a IL-13R-targeted immunotoxin has been administered in human clinical trials without apparent toxicity to normal tissues.

Nucleic acids encoding the IL-13Rα2 chain or vectors containing such nucleic acids can therefore be used for the manufacture of medicaments to introduce the IL-13Rα2 chain into cancer cells and thereby either inhibit their growth (for cells inhibited by the presence of the IL-13Rα2 chain) or to sensitize them to IL-13 immunoconjugates, or both. It is contemplated that to maximize the anti-tumor effect of the medicament, in most cases the practitioner will transfect tumor cells with the IL-13Rα2 chain and then administer an IL-13R-targeted immunoconjugate, such as an immunotoxin.

### Definitions

Units, prefixes, and symbols are denoted in their Systéme International de Unites (SI) accepted form. Numeric ranges are inclusive of the numbers defining the range. Unless otherwise indicated, nucleic acids are written left to right in 5′ to 3′ orientation; amino acid sequences are written left to right in amino to carboxy orientation. The headings provided herein are not limitations of the various aspects or embodiments of the invention which can be had by reference to the specification as a whole. Accordingly, the terms defined immediately below are more fully defined by reference to the specification in its entirety.

Interleukin 13 (“IL-13”) is an immunoregulatory protein produced by activated T helper-2 (“TH2”) cells that inhibits inflammation and monocyte differentiation, and upregulates MHC molecules on cell surfaces. IL-13 also induces the differentiation of dendritic cells from peripheral blood mononuclear cells. The protein encoded by the IL-13 cDNA is the human homologue of a mouse TH2 product called P600. IL-13 shares many of its biological activities with the TH2 cytokine Interleukin 4; both cytokines are able to enhance expression of CD23 on monocytes and B-cells and also induce IgE production. Production of many LPS-induced monokines, such as IL-1a, IL-1B, IL-6, IL-8, IL-10, TNFa, MIP-1a, GM-CSF and G-CSF are inhibited by IL-13, whereas IL-1ra is upregulated. These properties are shared with IL-4 and IL-10. In contrast to IL-4, IL-13 has no growth-promoting effect on T-cells and cannot compete for IL-4 binding to a human T-cell line. Therefore it was thought that a specific receptor for IL-13 is lacking on T-cells. More recently, however, an inhibitory effect of IL-13 on IL-8- and RANTES-induced chemotaxis of T-cells was described, indicating that T-cells do respond to IL-13, possibly by inhibition of production of the TH1 inducer IL-12.

The term “cpIL-13” is used to designate a circularly permuted (cp) IL-13. Circular permutation is functionally equivalent to taking a straight-chain molecule, fusing the ends (directly or through a linker) to form a circular molecule, and then cutting the circular molecule at a different location to form a new straight chain molecule with different termini.

The IL-13 receptor (“IL-13R”) is a heterodimeric molecule composed of two “chains” of approximately 65 kD proteins. The first chain is now known as the IL-13Rα1 chain, and was previously termed the IL-13Rα chain, or the IL-13α′ chain. An isoform was later cloned and was called IL-13Rα. To clarify references to the two forms of the chain, this isoform was then renamed as the IL-13α2 chain. As used herein, “IL-13α2” refers to this isoform. The amino acid sequence of the IL-13Rα2 chain (SEQ ID NO:1) and the native mRNA sequence encoding it (SEQ ID NO:2) were reported by Caput, D. et al., J. Biol. Chem. 271, 16921-16926 (1996); both sequences were deposited in GenBank under accession number X95302.

A “ligand”, as used herein, refers generally to all molecules capable of reacting with or otherwise recognizing or binding to a receptor on a target cell. Specifically, examples of ligands include, but are not limited to, antibodies, lymphokines, cytokines, receptor proteins such as CD4 and CD8, solubilized receptor proteins such as soluble CD4, hormones, growth factors, and the like which specifically bind desired target cells. In the context of the invention, the ligand will preferably be IL-13, a mutated IL-13 having a higher affinity for the IL-13α chain than wild-type IL-13, or a cpIL-13.

“Antibody” refers to a polypeptide ligand comprising at least a light chain or heavy chain immunoglobulin variable region which specifically recognizes and binds an epitope (e.g., an antigen). This includes intact immunoglobulins and the variants and portions of them well known in the art such as, Fab′ fragments, F(ab)′₂ fragments, single chain Fv proteins (“scFv”), and disulfide stabilized Fv proteins (“dsFv”). An scFv protein is a fusion protein in which a light chain variable region of an immunoglobulin and a heavy chain variable region of an immunoglobulin are bound by a linker. The term also includes genetically engineered forms such as chimeric antibodies (e.g., humanized murine antibodies), heteroconjugate antibodies (e.g., bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York (1997).

An antibody immunologically reactive with a particular antigen can be generated by recombinant methods such as selection of libraries of recombinant antibodies in phage or similar vectors, see, e.g., Huse, et al., Science 246:1275-1281 (1989); Ward, et al., Nature 341:544546 (1989); and Vaughan, et al., Nature Biotech. 14:309-314 (1996), or by immunizing an animal with the antigen or with DNA encoding the antigen.

The term “specifically deliver” as used herein refers to the preferential association of a molecule with a cell or tissue bearing a particular target molecule or marker and not to cells or tissues lacking that target molecule. It is, of course, recognized that a certain degree of non-specific interaction may occur between a molecule and a non-target cell or tissue. Nevertheless, specific delivery, may be distinguished as mediated through specific recognition of the target molecule. Typically specific delivery results in a much stronger association between the delivered molecule and cells bearing the target molecule than between the delivered molecule and cells lacking the target molecule. Specific delivery typically results in greater than 2 fold, preferably greater than 5 fold, more preferably greater than 10 fold and most preferably greater than 100 fold increase in amount of delivered molecule (per unit time) to a cell or tissue bearing the target molecule as compared to a cell or tissue lacking the target molecule or marker.

The term “residue” as used herein refers to an amino acid that is incorporated into a polypeptide. The amino acid may be a naturally occurring amino acid and, unless otherwise limited, may encompass known analogs of natural amino acids that can function in a similar manner as naturally occurring amino acids.

The terms “fusion protein,” “conjugate,” and “chimeric molecule” refer to a polypeptide formed by the joining of two or more polypeptides through a peptide bond formed between the amino terminus of one polypeptide and the carboxyl terminus of another polypeptide. The fusion protein may be formed by the chemical coupling of the constituent polypeptides or it may be expressed as a single polypeptide from nucleic acid sequence encoding the single contiguous fusion protein. A single chain fusion protein is a fusion protein having a single contiguous polypeptide backbone.

The terms “conjugating,” “joining,” “bonding” or “linking” refer to making two polypeptides into one contiguous polypeptide molecule, or to covalently attaching a radionuclide or other molecule to a polypeptide, such as an scFv. In the context of the present invention, the terms include reference to joining a ligand, such as an IL-13, a cpIL-13 or an antibody that specifically binds to the IL13α2 chain, to an effector molecule (“EM”). The linkage can be either by chemical or recombinant means. “Chemical means” refers to a reaction between the antibody moiety and the effector molecule such that there is a covalent bond formed between the two molecules to form one molecule.

As used herein, unless otherwise required by context, for convenience, the term “IL-13 conjugate” includes IL-13 conjugates, cpIL-13 conjugates, and anti-IL13α2 chain antibody-conjugates. For convenience of discussion, reference herein to an “IL-13-conjugate” means one targeted (for example, by IL-13 or by an antibody that specifically binds to the IL-13R) to the IL-13 receptor. Further, the art recognizes that immunotoxins or the like can be created as a fusion protein, or an effector molecule can be conjugated to IL-13 or another targeting molecule, such as an anti-IL-13R antibody, by chemical means. For ease of discussion herein, the terms “IL-13 conjugate,” “IL13R conjugate” and “IL-13R immunoconjugate” refer to both fusion proteins, such as IL-13-PE and its variants, and proteins chemically conjugated to an effector molecule, such as a radioisotope, unless otherwise required by context.

A “spacer” as used herein refers to a peptide that joins the proteins comprising a fusion protein. Generally a spacer has no specific biological activity other than to join the proteins or to preserve some minimum distance or other spatial relationship between them. However, the constituent amino acids of a spacer may be selected to influence some property of the molecule such as the folding, net charge, or hydrophobicity of the molecule.

The term “effector moiety” means the portion of a fusion protein or IL-13 conjugate, cpIL-13 conjugate, or anti-IL13α2 chain antibody-conjugate intended to have an effect on a cell targeted by the targeting moiety or to identify the presence of the conjugate. Thus, the effector moiety can be, for example, a therapeutic moiety, a toxin, a radiolabel, or a fluorescent label. In the context of the present invention, it is usually preferred that the effector moiety is a cytotoxin or a radioisotope (radioisotopes can be introduced into proteins or conjugated to an IL-13 or other targeting moiety by techniques well known in the art). A cytotoxin or other agent can be referred to as an effector molecule before it is conjugated to a targeting moiety and as an effector moiety thereafter, to emphasize that it is now part of a larger molecule. For convenience, however, persons in the art sometimes continue to refer to a conjugated cytotoxin or other effector moiety as an “effector molecule.” Unless otherwise required by context, therefore, the terms “effector moiety” and “effector molecule” are used synonymously herein, and both are represented by the term “EM.”

A “toxic moiety” is the portion of an IL-13 conjugate which renders the conjugate cytotoxic to cells of interest.

A “therapeutic moiety” is the portion of an IL-13 conjugate intended to act as a therapeutic agent.

The term “therapeutic agent” includes any number of compounds currently known or later developed to act as anti-neoplastics, anti-inflammatories, cytokines, anti-infectives, enzyme activators or inhibitors, allosteric modifiers, antibiotics or other agents administered to induce a desired therapeutic effect in a patient. The therapeutic agent may also be a toxin or a radioisotope.

The terms “effective amount” or “amount effective to” or “therapeutically effective amount” includes reference to a dosage of a therapeutic agent sufficient to produce a desired result, such as inhibiting cell protein synthesis by at least 50%, or killing the cell.

The terms “toxin” or “cytotoxin” include reference to abrin, ricin, Pseudomonas exotoxin (PE), diphtheria toxin (DT), botulinum toxin, or modified versions thereof that retain activity as cytotoxins. For example, PE and DT are highly toxic compounds that typically bring about death through liver toxicity. PE and DT, however, can be modified into a form for use as an immunotoxin by removing the native targeting component of the toxin (e.g., domain Ia of PE or the B chain of DT) and replacing it with a different targeting moiety, such as an antibody. Additional mutations and deletions can also be made, typically to decrease the size of the molecule to enhance its ability to penetrate solid tumors.

The term “contacting” includes reference to placement in direct physical association.

An “expression plasmid” comprises a nucleotide sequence encoding a molecule or interest, which is operably linked to a promoter.

Conventional notation is used herein to portray polypeptide sequences: the left-hand end of a polypeptide sequence is the amino-terminus; the right-hand end of a polypeptide sequence is the carboxyl-terminus.

“Fusion protein” refers to a polypeptide formed by the joining of two or more polypeptides through a peptide bond formed by the amino terminus of one polypeptide and the carboxyl terminus of the other polypeptide. A fusion protein may is typically expressed as a single polypeptide from a nucleic acid sequence encoding the single contiguous fusion protein. However, a fusion protein can also be formed by the chemical coupling of the constituent polypeptides.

“Conservative substitution” refers to the substitution in a polypeptide of an amino acid with a functionally similar amino acid. The following six groups each contain amino acids that are conservative substitutions for one another.

1) Alanine (A), Serine (S), Threonine Cr);

2) Aspartic acid (D), Glutamic acid (E);

3) Asparagine (N), Glutatine (Q);

4) Arginine (R), Lysine (K);

5) Isoleucine (I), Leucine (L), Methionine (M), Valine (V); and

6) Phenylalanine (F), Tyrosine (Y), Tryptophan (W).

See also, Creighton, PROTEINS, W.H. Freeman and Company, New York (1984).

Two proteins are “homologs” of each other if they exist in different species, are derived from a common genetic ancestor and share at least 70% amino acid sequence identity.

“Substantially pure” or “isolated” means an object species is the predominant species present (i.e., on a molar basis, more abundant than any other individual macromolecular species in the composition), and a substantially purified fraction is a composition wherein the object species comprises at least about 50% (on a molar basis) of all macromolecular species present. Generally, a substantially pure composition means that about 80% to 90% or more of the macromolecular species present in the composition is the purified species of interest. The object species is purified to essential homogeneity (contaminant species cannot be detected in the composition by conventional detection methods) if the composition consists essentially of a single macromolecular species. Solvent species, small molecules (<500 Daltons), stabilizers (e.g., BSA), and elemental ion species are not considered macromolecular species for purposes of this definition.

“Nucleic acid” refers to a polymer composed of nucleotide units (ribonucleotides, deoxyribonucleotides, related natally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. Such polynucleotides can be synthesized, for example, using an automated DNA synthesizer. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand having the same sequence as an mRNA transcribed from that DNA and which are located 5′ to the 5′-end of the RNA transcript are referred to as “upstream sequences”; sequences on the DNA strand having the same sequence as the RNA and which are 3′ to the 3′ end of the coding RNA transcript are referred to as “downstream sequences.”

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (ie., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Recombinant nucleic acid” refers to a nucleic acid having nucleotide sequences that are not naturally joined together. This includes nucleic acid vectors comprising an amplified or assembled nucleic acid which can be used to transform a suitable host cell. A host cell that comprises the recombinant nucleic acid is referred to as a “recombinant host cell.” The gene is then expressed in the recombinant host cell to produce, e.g., a “recombinant polypeptide.” A recombinant nucleic acid may serve a non-coding function (e.g., promoter, origin of replication, ribosome-binding site, etc.) as well.

“Expression control sequence” refers to a nucleotide sequence in a polynucleotide that regulates the expression (transcription and/or translation) of a nucleotide sequence operatively linked thereto. “Operatively linked” refers to a functional relationship between two parts in which the activity of one part (e.g., the ability to regulate transcription) results in an action on the other part (e.g., transcription of the sequence). Expression control sequences can include, for example and without limitation, sequences of promoters (e.g., inducible or constitutive), enhancers, transcription terminators, a start codon (ie., ATG), splicing signals for introns, and stop codons.

“Expression cassette” refers to a recombinant nucleic acid construct comprising an expression control sequence operatively linked to an expressible nucleotide sequence. An expression cassette generally comprises sufficient cis-acting elements for expression; other elements for expression can be supplied by the host cell or in vitro expression system.

“Expression vector” refers to a vector comprising an expression cassette. Expression vectors include all those known in the art, such as cosmids, plasmids (e.g., naked or contained in liposomes) and viruses that incorporate the expression cassette.

A first sequence is an “antisense sequence” with respect to a second sequence if a polynucleotide whose sequence is the first sequence specifically hybridizes with a polynucleotide whose sequence is the second sequence.

Terms used to describe sequence relationships between two or more nucleotide sequences or amino acid sequences include “reference sequence,” “selected from,” “comparison window,” “identical,” “percentage of sequence identity,” “substantially identical,” “complementary,” and “substantially complementary.”

For sequence comparison of nucleic acid sequences, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are used. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds 1995 supplement)).

One example of a useful algorithm is PILEUP. PILEUP uses a simplification of the progressive alignment method of Feng & Doolittle, J. Mol. Evol. 35:351-360 (1987). The method used is similar to the method described by Higgins & Sharp, CABIOS 5:151-153 (1989). Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc. Acids Res. 12:387-395 (1984).

Another example of algorithms that are suitable for determining percent sequence identity and sequence similarity are the BLAST and the BLAST 2.0 algorithm, which are described in Altschul et al., J. Mol. Biol. 215:403410 (1990) and Altschul et al., Nucleic Acids Res. 25:3389-3402 (1977)). Software for performing BLAST analyses is publicly available through the National Center for Biotechnology Information (http://www.ncbi.nlm.nih.gov/). The BLASTN program (for nucleotide sequences) uses as defaults a word length (W) of 11, alignments (B) of 50, expectation (E) of 10, M=5, N-4, and a comparison of both strands. The BLASTP program (for amino acid sequences) uses as defaults a word length (W) of 3, and expectation (E) of 10, and the BLOSUM62 scoring matrix (see Henikoff & Henikoff, Proc. Natl. Acad. Sci. USA 89:10915 (1989)).

“Stringent hybridization conditions” refers to 50% formamide, 5×SSC and 1% SDS incubated at 42° C. or 5×SSC and 1% SDS incubated at 65° C., with a wash in 0.2×SSC and 0.1% SDS at 65° C.

“Naturally-occurring” as applied to an object refers to the fact that the object can be found in nature. For example, an amino acid or nucleotide sequence that is present in an organism (including viruses) that can be isolated from a source in nature and which has not been intentionally modified by man in the laboratory is naturally-occurring.

### The IL-13Rα2 Chain

The amino acid sequence of the IL-13Rα2 chain (SEQ ID NO:1) and the native mRNA sequence encoding it (SEQ ID NO:2) were reported by Caput, D. et al., J. Biol. Chem. 271, 16921-16926 (1996); both sequences were deposited in GenBank under accession number X95302. Persons of skill in the art will recognize that, due to the degeneracy of the genetic code, numerous nucleic acid sequences other than SEQ ID NO:2 could be constructed which code for the same amino acid sequence. Such sequences are encompassed within the scope of the present invention. They can, for example, be substituted for the native sequence in transfecting cancer cells.

Persons of skill will also appreciate that the native amino acid sequence of the IL-13Rα2 chain can be subjected to a number of changes and still bind IL-13. Studies in the early 1990's of the lac repressor, for example, established that proteins are surprisingly tolerant to amino acid substitutions, with half of the amino acid substitutions made being phenotypically silent in the function of the protein. It is anticipated that substitutions (and especially conservative substitutions) and other changes can be made in the amino acid sequence of SEQ ID NO:1 that result in a polypeptide that can still bind IL-13 and which can be used in place of the native IL-13α2 chain in the methods herein.

A review of the sequence of the IL-13Rα2 chain to other proteins indicated the greatest identity was with- regard to the a chain of the IL-5 receptor, with which the IL-13Rα2 chain had a 51% identity. In the practice ofthis invention, it is preferred that the tumor cells be transfected with a nucleic acid sequence encoding a polypeptide with about 70% or greater identity to SEQ ID NO:1 and which can specifically bind IL-13. More preferably, the nucleic acid encodes a polypeptide with about 75% or greater identity to SEQ ID NO:1 and which can specifically bind IL-13. Still more preferably, the nucleic acid encodes a polypeptide with about 80% or greater identity to SEQ ID NO:1 and which can specifically bind IL-13. More preferably yet, the nucleic acid encodes a polypeptide with about 85% or greater identity to SEQ ID NO:1 and which can specifically bind IL-13. Even more preferably, the nucleic acid encodes a polypeptide with about 90% or greater identity to SEQ ID NO:1 and which can specifically bind IL-13. Yet more preferably, the nucleic acid encodes a polypeptide with about 95% or greater identity to SEQ ID NO:1 and which can specifically bind IL-13. Most preferably, the nucleic acid has the sequence of SEQ ID NO:1.

Conveniently, the nucleic acid sequence encoding the IL-13Rα2 chain is in an expression vector in which the coding sequence is operatively linked to a promoter which will drive expression in the cells of interest. In some embodiments, the promoter is specific for the tissue or organ of the tumor (for example, a prostate-specific promoter if the target cancer is a prostate cancer) to promote expression of the nucleic acid in cells of the tumor.

### Sensitization of Cancer Cells to Immunoconjugates

Once the cells have been transfected with IL-13Rα2 chain, they are sensitized to immunoconjugates targeted by IL-13 or by antibodies to the IL-13Rα2 chain. The immunoconjugate can comprise a therapeutic molecule, or a toxic moiety, which can be, for example, a radioisotope or a toxin. In preferred embodiments, the toxin is a Pseudomonas exotoxin A (“PE”) which has been modified to reduce or to eliminate non-specific binding. A variety of such mutated PE molecules are known in the art, as discussed further herein.

In the past several years, investigations have been made on approaches in which cancer cells have been sensitized to benign prodrugs that become cytotoxic to cells transfected with nucleic acids encoding an enzyme that has no direct effects on cellular function. The enzyme expression confers toxicity to an otherwise benign compound that brings about cell death. In the exemplary demonstration of the approach, the herpes simplex virus thymidine kinase (HSV-tk) gene was transferred into cancer cells and normal cells followed by treatment with the anti-herpes drugs acyclovir or ganciclovir. Selective cell death of transduced cells was shown in vitro and in vivo (Heyman et al., Proc Natl Acad Sci USA, 86:2698-2702 (1989)). In another approach, a bacterial and fungal enzyme, cytosine deaminase, (CDA) which catalyzes hydrolytic deamination of cytosine to uracil was introduced into cancer cells. Cells that express CDA convert 5-fluorocytocine (5-FC), a fungicidal and bactericidal drug, to 5-fluorouracil (5-FU), which is then phosphorylated and subsequently inhibits gene transcription, resulting in cell death (Mullen et al., Proc Natl Acad Sci USA. 89:33-37 (1992)). Although animal models showed remarkable antitumor activity for the HSV-tk approach, clinical results in a phase I clinical trial for the treatment of malignant glioma (Ram et al., Nat Med., 3:1354-1361 (1997)) were not as great as hoped. These studies may have been hampered by poor transfection or gene transfer of the nucleic acids into tumor cells in the human trials by the use for viral vectors. Viral vectors are comparatively large and have difficulty penetrating beyond the superficial layer of cells in the tumor bed of the solid tumors treated. In most studies, the viral vectors utilized had limited distribution within the tumor bed even after intratumoral administration (Ram et al., Nat Med., supra.)

The present invention, however, does not rely on the mechanism for killing cancer cells used in the HSV-tk studies. The results reported in the Examples herein in the animal models for head and neck cancers and for prostate cancer suggest that even limited transfection of tumor cells with the IL-13Rα2 chain, followed by the administration of an IL-13-targeted immunotoxin, results in a reduction or even a remission of the entire tumor. Moreover, the methods of delivering nucleic acids to cells, in particular, the use of liposomal delivery systems or direct introduction of nucleic acids into cells of the tumor by gene guns or biolistic methods, should provide a transfection of cells deeper into the tumor bed than may have been accomplished in the HSV-tk or CDA studies. Thus, the present invention solves some of the problems seen with the HSV-tk approach and the CDA approach.

Nonetheless, it is expected that the results obtained with transfecting cancer cells with the IL-13Rα2 chain can be flirter improved by increasing the proportion of cells that are transfected with the chain. This can be accomplished, for example, by encapsulating the plasmid in liposomes for better distribution and gene transfer within the tumor bed and by using tissue specific promoters can be used so that direct IL-13Rα2 gene expression will occur only in the specific tissues with tumor; so that cell death after IL-13 cytotoxin therapy will be limited to the target tissues. Finally, as IL-13R are present in low levels on some cancer cells, upregulation of IL-13R expression can be achieved by use of common pharmacological agents (e.g., steroids and cytokines) to render the cancer cells more sensitive to IL-13 targeted therapy even without the use of in vivo gene transfer.

### Production of Immunoconjugates

Immunoconjugates include, but are not limited to, molecules in which there is a covalent linkage of a therapeutic agent to an antibody. A therapeutic agent is an agent with a particular biological activity directed against a particular target molecule or a cell bearing a target molecule. One of skill in the art will appreciate that therapeutic agents may include various drugs such as vinblastine, daunomycin and the like, cytotoxins such as native or modified Pseudomonas exotoxin or diphtheria toxin, encapsulating agents, (e.g., liposomes) which themselves contain pharmacological compositions, radioactive agents such as ¹²⁵I, ³²P, ¹⁴C, ³H and ³⁵S and other labels, target moieties and ligands. IL-13 receptor-specific chimeric proteins that can be used to target cancer cells after transfection with the IL-13Rα2 chain are described, for example, Puri et al., U.S. Pat. No. 5,919,456, Puri et al., U.S. Pat. No. 5,614,191. Methods of circularly permutating cytokines other than IL-13 are described, for example, in U.S. Pat. Nos. 5,635,599 and 6,011,002. Mutants of IL-13 that can be used as targeting moieties are described in, e.g., WO 01/25282 and WO 99/51643.

The choice of a particular therapeutic agent depends on the particular target molecule or cell and the biological effect is desired to evoke. Thus, for example, the therapeutic agent may be a cytotoxin which is used to bring about the death of a particular target cell. Conversely, where it is merely desired to invoke a non-lethal biological response, the therapeutic agent may be conjugated to a non-lethal pharmacological agent or a liposome containing a non-lethal pharmacological agent.

With the therapeutic agents and antibodies herein provided, one of skill can readily construct a variety of clones containing functionally equivalent nucleic acids, such as nucleic acids which differ in sequence but which encode the same EM or antibody sequence. Thus, the present invention provides nucleic acids encoding antibodies and conjugates and fusion proteins thereof

A. Recombinant and Synthetic Methods of Producing Immunoconjugates

Nucleic acid sequences encoding the chimeric molecules of the present invention can be prepared by any suitable method including, for example, cloning of appropriate sequences or by direct chemical synthesis by methods such as the phosphotriester method of Narang, et al., Meth. Enzymol. 68:90-99 (1979); the phosphodiester method of Brown, et al., Meth. Enzymol. 68:109-151 (1979); the diethylphosphoramidite method of Beaucage, et al., Tetra. Lett. 22:1859-1862 (1981); the solid phase phosphoramidite triester method described by Beaucage & Caruthers, Tetra. Letts. 22(20):1859-1862 (1981), e.g., using an automated synthesizer as described in, for example, Needham-VanDevanter, et al. Nuci. Acids Res. 12:6159-6168 (1984); and, the solid support method of U.S. Pat. No. 4,458,066. Chemical synthesis produces a single stranded oligonucleotide. This may be converted into double stranded DNA by hybridization with a complementary sequence, or by polymerization with a DNA polymerase using the single strand as a template. One of skill would recognize that while chemical synthesis of DNA is limited to sequences of about 100 bases, longer sequences may be obtained by the ligation of shorter sequences.

In a preferred embodiment, the nucleic acid sequences of this invention are prepared by cloning techniques. Examples of appropriate cloning and sequencing techniques, and instructions sufficient to direct persons of skill through many cloning exercises are found in Sambrook, et al., supra, Berger and Kimmel (eds.), supra, and Ausubel, supra. Product information from manufacturers of biological reagents and experimental equipment also provide useful information. Such manufacturers include the SIGMA chemical company (Saint Louis, Mo.), R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersberg, Md.), Fluka Chemica-Biochemika Analytika Fluka Chemie AG, Buchs, Switzerland), Inyitrogen, San Diego, Calif., and Applied Biosystems (Foster City, Calif.), as well as many other commercial sources known to one of skill.

Nucleic acids encoding native EM or anti-IL-13Rα chain antibodies can be modified to form the EM, antibodies, or immunoconjugates of the present invention. Modification by site-directed mutagenesis is well known in the art. Nucleic acids encoding EM or anti-IL-13Rα chain antibodies can be amplified by in vitro methods. Amplification methods include polymerase chain reaction (PCR), the ligase chain reaction (LCR), the transcription-based amplification system (TAS), the self-sustained sequence replication system (3SR). A wide variety of cloning methods, host cells, and in vitro amplification methodologies are well known to persons of skill.

In a preferred embodiment, immunoconjugates are prepared by inserting the cDNA which encodes an anti-IL-13Rα chain scFv antibody into a vector which comprises the cDNA encoding the EM. The insertion is made so that the scFv and the EM are read in frame, that is in one continuous polypeptide which contains a functional Fv region and a functional EM region. In a particularly preferred embodiment, cDNA encoding a diphtheria toxin fragment is ligated to a scFv so that the toxin is located at the carboxyl terminus of the scFv. In a most preferred embodiment, cDNA encoding PE is ligated to a scFv so that the toxin is located at the amino terminus of the scFv.

Once the nucleic acids encoding an EM, anti-IL-13Rα chain antibody, or an immunoconjugate of the present invention are isolated and cloned, one may express the desired protein in a recombinantly engineered cell such as bacteria, plant, yeast, insect and mammalian cells. It is expected that those of skill in the art are knowledgeable in the numerous expression systems available for expression of proteins including E. coli, other bacterial hosts, yeast, and various higher eucaryotic cells such as the COS, CHO, HeLa and myeloma cell lines. No attempt to describe in detail the various methods known for the expression of proteins in prokaryotes or eukaryotes will be made.

One of skill would recognize that modifications can be made to a nucleic acid encoding a polypeptide of the present invention (i e., anti- IL-13Rα chain antibody, PE, or an immunoconjugate formed from their combination) without diminishing its biological activity. Some modifications may be made to facilitate the cloning, expression, or incorporation of the targeting molecule into a fusion protein. Such modifications are well known to those of skill in the art and include, for example, termination codons, a methionine added at the amino terminus to provide an initiation, site, additional amino acids placed on either terminus to create conveniently located restriction sites, or additional amino acids (such as poly His) to aid in purification steps.

In addition to recombinant methods, the immunoconjugates, EM, and antibodies of the present invention can also be constructed in whole or in part using standard peptide synthesis. Solid phase synthesis of the polypeptides of the present invention of less than about 50 amino acids in length may be accomplished by attaching the C-terminal amino acid of the sequence to an insoluble support followed by sequential addition of the remaining amino acids in the sequence. Techniques for solid phase synthesis are described by Barany & Merrifield, THE PEPTIDES: ANALYSIS, SYNTHESIS, BiOLOGY. VOL. 2: SPECIAL METHODS IN PEPTIDE SYNTHESIS, PART A. pp. 3-284; Merrifield, et al. J. Am. Chem. Soc. 85:2149-2156 (1963), and Stewart, et al., SOLID PHASE PEPTIDE SYNTHESIS, 2ND ED., Pierce Chem. Co., Rockford, Ill. (1984). Proteins of greater length may be synthesized by condensation of the amino and carboxyl termini of shorter fragments. Methods of forming peptide bonds by activation of a carboxyl terminal end (e.g., by the use of the coupling reagent N, N′-dicycylohexylcarbodiimide) are known to those of skill.

In some embodiments, the targeting molecule (whether recombinantly or synthetically made) is chemically conjugated to the effector molecule (e.g. a cytotoxin, a label, a ligand, or a drug or liposome). Means of chemically conjugating molecules are well known to those of skill and are set forth in standard texts, such as Hermanson, Bioconjugate Techniques, Academic Press San Diego, Calif. (1996). The procedure for attaching an agent to an antibody or other polypeptide targeting molecule will vary according to the chemical structure of the agent. Polypeptides typically contain variety of functional groups; e.g., carboxylic acid (COOH) or free amine (—NH₂) groups, which are available for reaction with a suitable functional group on an effector molecule to bind the effector thereto. Alternatively, the targeting molecule and/or effector molecule may be derivatized to expose or attach additional reactive functional groups. The derivitization may involve attachment of any of a number of linker molecules such as those available from Pierce Chemical Company, Rockford Ill.

A “linker”, as used herein, is a molecule that is used to join the antibody to the effector molecule (once joined, the previously separate antibody and effector molecules are sometimes referred to as the targeting moiety and the effector moiety of the immunoconjugate, respectively). The linker is capable of forming covalent bonds to both the antibody and to the effector molecule. Suitable linkers are well known to those of skill in the art and include, but are not limited to, straight or branched-chain carbon linkers, heterocyclic carbon linkers, or peptide linkers. Where the antibody and the effector molecule are polypeptides, the linkers may be joined to the constituent amino acids through their side groups (e.g., through a disulfide linkage to cysteine). However, in a preferred embodiment, the linkers will be joined to the alpha carbon amino and carboxyl groups of the terminal amino acids.

In some circumstances, it is desirable to free the effector molecule from the antibody when the immunoconjugate has reached its target site. Therefore, in these circumstances, immunoconjugates will comprise linkages which are cleavable in the vicinity of the target site. Cleavage of the linker to release the effector molecule from the antibody may be prompted by enzymatic activity or conditions to which the immunoconjugate is subjected either inside the target cell or in the vicinity of the target site. When the target site is a tumor, a linker which is cleavable under conditions present at the tumor site (e.g. when exposed to tumor-associated enzymes or acidic pH) may be used.

B. Purification

Once expressed, the recombinant immunoconjugates, antibodies, and/or effector molecules of the present invention can be purified according to standard procedures of the art, including ammonium sulfate precipitation, affinity columns, column chromatography, and the like (see, generally, R Scopes, PROTEIN PURIFICATION, Springer-Verlag, New York (1982)). Substantially pure compositions of at least about 90 to 95% homogeneity are preferred, and 98 to 99% or more homogeneity are most preferred for pharmaceutical uses. Once purified, partially or to homogeneity as desired, if to be used therapeutically, the polypeptides should be substantially free of endotoxin.

Methods for expression of single chain antibodies and/or refolding to an appropriate active form, including single chain antibodies, from bacteria such as E. coli have been described and are well-known and are applicable to the antibodies of this invention. See, Buchner, et al., Anal. Biochem. 205:263-270 (1992); Pluckthun, Biotechnology 9:545 (1991); Huse, et al., Science 246:1275 (1989) and Ward, et al., Nature 341:544 (1989), all incorporated by reference herein.

Often, functional heterologous proteins from E. coli or other bacteria are isolated from inclusion bodies and require solubilization using strong denaturants, and subsequent refolding. During the solubilization step, as is well-known in the art, a reducing agent must be present to separate disulfide bonds. An exemplary buffer with a reducing agent is: 0.1 M Tris pH 8, 6 M guanidine, 2 mM EDTA, 0.3 M DTE (dithioerythritol). Reoxidation of the disulfide bonds can occur in the presence of low molecular weight thiol reagents in reduced and oxidized form, as described in Saxena, et al., Biochemistry 9: 5015-5021 (1970), incorporated by reference herein, and especially as described by Buchner, et al., supra.

Renaturation is typically accomplished by dilution (e.g., 100-fold) of the denatured and reduced protein into refolding buffer. An exemplary buffer is 0.1 M Tris, pH 8.0,0.5 M L-arginine, 8 mM oxidized glutathione (GSSG), and 2 mM EDTA.

As a modification to the two chain antibody purification protocol, the heavy and light chain regions are separately solubilized and reduced and then combined in the refolding solution. A preferred yield is obtained when these two proteins are mixed in a molar ratio such that a 5 fold molar excess of one protein over the other is not exceeded. It is desirable to add excess oxidized glutathione or other oxidizing low molecular weight compounds to the refolding solution after the redox-shuffling is completed.

### Cytotoxins for Use in Immunotoxins

Toxins can be employed with antibodies of the present invention to yield chimeric molecules, such as immunotoxins. Exemplary toxins include ricin, abrin, diphtheria toxin and subunits thereof, ribotoxin, ribonuclease, saporin, and calicheamicin, as well as botulinum toxins A through F. These toxins are well known in the art and many are readily available from commercial sources (e.g., Sigma Chemical Company, St. Louis, Mo.). Diphtheria toxin is isolated from Corynebacterium diphtheriae. Ricin is the lectin RCA60 from Ricinus communis (Castor bean). The term also references toxic variants thereof For example, see, U.S. Pat. Nos. 5,079,163 and 4,689,401. Ricinus communis agglutinin (RCA) occurs in two forms designated RCA₆₀ and RCA₁₂₀ according to their molecular weights of approximately 65 and 120 kD, respectively (Nicholson & Blaustein, J. Biochim. Biophys. Acta 266:543 (1972)). The A chain is responsible for inactivating protein synthesis and killing cells. The B chain binds ricin to cell-surface galactose residues and facilitates transport of the A chain into the cytosol (Olsnes, et al., Nature 249:627-631 (1974) and U.S. Pat. No.3,060,165). Conjugating ribonucleases to targeting molecules for use as immunotoxins is discussed in, e.g., Suzuki et al., Nat Biotech 17:265-70 (1999). Exemplary ribotoxins such as α-sarcin and restrictocin are discussed in, e.g., Rathore et al., Gene 190:31-5 (1997) and Goyal and Batra, Biochem 345 Pt 2:247-54 (2000). Calicheamicins were first isolated from Micromonospora echinospora and are members of the enediyne antitumor antibiotic family that cause double strand breaks in DNA that lead to apoptosis. See, e.g., Lee et al., J. Antibiot 42:1070-87 (1989). The drug is the toxic moiety of an immunotoxin in clinical trials. See, e.g., Gillespie et al., Ann Oncol 11:73541 (2000).

Abrin includes toxic lectins from Abrus precatorius. The toxic principles, abrin a, b, c, and d, have a molecular weight of from about 63 and 67 kD and are composed of two disulfide-linked polypeptide chains A and B. The A chain inhibits protein synthesis; the B-chain (abrin-b) binds to D-galactose residues (see, Funatsu, et al., Agr. Biol. Chem. 52:1095 (1988); and Olsnes, Methods Enzymol. 50:330-335 (1978)).

In preferred embodiments of the present invention, the toxin is Pseudomonas exotoxin (PE). The term “Pseudomonas exotoxin” as used herein refers to a full-length native (naturally occurring) PE or a PE that has been modified. Such modifications may include, but are not limited to, elimination of domain Ia, various amino acid deletions in domains Ib, II and III, single amino acid substitutions and the addition of one or more sequences at the carboxyl terminus such as KDEL and REDL. See Siegall, et al., J. Biol. Chem. 264:14256-14261 (1989). In a preferred embodiment, the cytotoxic fragment of PE retains at least 50%, preferably 75%, more preferably at least 90%, and most preferably 95% of the.cytotoxicity of native PE. In a particularly preferred embodiment, the cytotoxic fragment is more toxic than native PE.

Native Pseudomonas exotoxin A (“PE”) is an extremely active monomeric protein (molecular weight 66 kD), secreted by Pseudomonas aeruginosa, which inhibits protein synthesis in eukaryotic cells. The native PE sequence is provided in commonly assigned U.S. Pat. No. 5,602,095, incorporated herein by reference. The method of action is inactivation of the ADP-ribosylation of elongation factor 2 (EF-2). The exotoxin contains three structural domains that act in concert to cause cytotoxicity. Domain Ia (amino acids 1-252) mediates cell binding. Domain II (amino acids 253-364) is responsible for translocation into the cytosol and domain II (amino acids 400613) mediates ADP ribosylation of elongation factor 2. The function of domain Ib (amino acids 365-399) remains undefined, although a large part of it, amino acids 365-380, can be deleted without loss of cytotoxicity. See Siegall, et al., (1989), supra.

PE employed in the present invention include the native sequence, cytotoxic fragments of the native sequence, and conservatively modified variants of native PE and its cytotoxic fragments. Cytotoxic fragments of PE include those which are cytotoxic with or without subsequent proteolytic or other processing in the target cell (e.g., as a protein or pre-protein). Cytotoxic fragments of PE known in the art include PE40, PE38, and PE35.

In preferred embodiments, the PE has been modified to reduce or eliminate non-specific cell binding, frequently by deleting domain Ia as taught in U.S. Pat. No. 4,892,827, although this can also be achieved, for example, by mutating certain residues of domain Ia U.S. Pat. No. 5,512,658, for instance, discloses that a mutated PE in which Domain Ia is present but in which the basic residues of domain Ia at positions 57, 246, 247, and 249 are replaced with acidic residues (glutamic acid, or “E”)) exhibits greatly diminished non-specific cytotoxicity. This mutant form of PE is sometimes referred to as PE4E.

PE40 is a truncated derivative of PE as previously described in the art. See, Pai, et al., Proc. Nat'l. Acad. Sci. USA 88:3358-62(1991); and Kondo, et al., J. Biol. Chem. 263:9470-9475 (1988). PE35 is a 35 kD carboxyl-terminal fragment of PE in which amino acid residues 1-279 have deleted and the molecule commences with a met at position 280 followed by amino acids 281-364 and 381-613 of native PE. PE35 and PE40 are disclosed, for example, in U.S. Pat. Nos. 5,602,095 and 4,892,827.

In some preferred embodiments, the cytotoxic fragment PE38is employed. PE38 is a truncated PE pro-protein composed of amino acids 253-364 and 381-613 which is activated to its cytotoxic form upon processing within a cell (see e.g., U.S. Pat. No. 5,608,039, and Pastan et al., Biochin. Biophys. Acta 1333:C1-C6 (1997)).

While in preferred embodiments, the PE is PE4E, PE40, or PE38, any form of PE in which non-specific cytotoxicity has been eliminated or reduced to levels in which significant toxicity to non-targeted cells does not occur can be used in the immunotoxins of the present invention so long as it remains capable of translocation and EF-2 ribosylation in a targeted cell.

In a preferred embodiment, the IL-13Rα chain-targeted cytotoxins of this invention comprise the PE molecule designated PE4E. PE4E is a “full length” PE with a mutated and inactive native binding domain where amino acids 57, 246, 247, and 249 are all replaced by glutamates (see, e.g., Chaudhary et al., J. Biol. Chem., 265: 16306 (1995)).

In another preferred embodiment, the IL-13Rα chain-targeted cytotoxins of this invention comprise the PE molecule designated PE38. This PE molecule is a truncated form of PE composed of amino acids 253-364 and 381-608. One preferred modification of PE38 is to modify the carboxyl terminus to KDEL to form PE38KDEL. In some studies, good results was obtained with a variant of PE38 termed PE38QQR, in which the lysine residues at positions 509 and 606 are replaced by glutamnine and one at position 613 is replaced by arginine (Debinski et al. Bioconj. Chem., 5: 40 (1994)). In further studies, however, no difference was seen between the toxicity of immunotoxins employing PE38QQR as the toxic moiety and those employing PE38.

A. Conservatively Modified Variants of PE

Conservatively modified variants of PE or cytotoxic fragments thereof have at least 80% sequence similarity, preferably at least 85% sequence similarity, more preferably at least 90% sequence similarity, and most preferably at least 95% sequence similarity at the amino acid level, with the PE of interest, such as PE38.

The term “conservatively modified variants” applies to both amino acid and nucleic acid sequences. With respect to particular nucleic acid sequences, conservatively modified variants refer to those nucleic acid sequences which encode identical or essentially identical amino acid sequences, or if the nucleic acid does not encode an amino acid sequence, to essentially identical nucleic acid sequences. Because of the degeneracy of the genetic code, a large number of functionally identical nucleic acids encode any given polypeptide. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid.

B. Assaying for Cytotoxicity of PE

Pseudomonas exotoxins employed in the invention can be assayed for the desired level of cytotoxicity by assays well known to those of skill in the art. Exemplary toxicity assays are described herein at, e.g., Example 2. Thus, cytotoxic fragments of PE and conservatively modified variants of such fragments can be readily assayed for cytotoxicity. A large number of candidate PE molecules can be assayed simultaneously for cytotoxicity by methods well known in the art. For example, subgroups of the candidate molecules can be assayed for cytotoxicity. Positively reacting subgroups of the candidate molecules can be continually subdivided and reassayed until the desired cytotoxic fragment(s) is identified. Such methods allow rapid screening of large numbers of cytotoxic fragments or conservative variants of PE.

### EXAMPLES

The following examples are offered to illustrate, but not to limit the claimed invention.

### Example 1

This Example sets forth the results of studies demonstrating that cancer cells transfected with IL-13α2 chain become susceptible to immunoconjugates bearing targeting moieties to IL-13 receptors.

A. Materials and Methods

Recombinant Cytokines and Toxins

Recombinant human IL-4 and IL-13 were produced and purified to near homogeneity. The cpIL4-toxin IL4(38-37)-PE38DEL, containing the circularly permuted IL-4 mutant in which amino acids 38-129 were linked to amino acids 1-37 via a GGNGG linker and then fused to truncated toxin PE38KDEL, consisting of amino acids 253-364 and 381-608 of Pseudomonas exotoxin (PE) followed by KDEL, was expressed in Escherichia coli and purified as described previously (Kreitman, R. J. et al., Proc. Natl. Acad. Sci. U.S.A. 91, 6889-6893 (1994); Kreitman, R. J. et al., Cancer Res. 55, 3357-3363 (1995); Puri, R. K. et al., Cancer Res. 56, 5631-5637 (1996)).

Cell Lines

The human glioblastoma multiforme cell line T98G, head and cancer cell line A253, renal cell cancer cell line Caki-1, and pancreatic cancer cell line PANC-1 were purchased from the American Type Culture Collection (Rockville, Md.). These cell lines were cultured in EMEM (T98G), McCoy's 5A (A253 and Caki-1), or DMEM (PANC-1) containing 10% fetal bovine serum (BioWhittaker; Walkersville, Md.), 1 mM HEPES, 1 mM L-glutamine, penicillin (100 μg/ml), and streptomycin (100 μg/ml) (BioWhittaker).

Plasmids and Transient Transfection of DNA

cDNA encoding human IL-13Rα chain (Caput, D. et al., J. Biol. Chem. 271, 16921-16926 (1996)) was cloned into pME18S mammalian expression vector. Plasmid DNA (6 μg/60-mm dish or 12 μg/100-mm culture dish) was transfected into semiconfluent cells by GenePORTER transfection reagent (Gene Therapy Systems, San Diego, Calif.) according to the manufacturer instructions. Briefly, cells (1×10⁶/60-mm dish or 3×10⁶/100-mm dish) were cultured with DNA-GenePORTER mixture for 5 hr in DMEM. DMEM containing 20% FBS was then added and the culture continued for an additional 24 hr after transfection. The medium was then changed and cells were cultured for a final 24-hr period.

Radioreceptor Binding

Recombinant human IL-13 was labeled with ¹²⁵I (Amersham, Arlington Heights, Ill.) using the IODO-GEN reagent (Pierce, Rockford, Ill.) as described (Obiri, N. I. et al., J. Biol. Chem. 270, 8797-8804 (1995)). The specific activity of the radiolabeled IL-13 was estimated to be 12.7 μCi/μg of protein. For binding experiments, 5×10⁵ cells in 100 μl of binding buffer (RPMI 1640 containing 0.2% human serum albumin and 10 mM HEPES) were incubated with 200 pM¹²⁵ I-labeled IL-13 (¹²⁵IL-13) with or without 40 nM/unlabeled IL-4 or IL-13 at 4° C. for 2 hr. Cell-bound ¹²⁵I-IL-13 was separated from unbound centrifugation through a phthalate oil gradient and radioactivity was determined with a γ counter (Wallac, Gaithersburg, Md.).

Protein Synthesis Inhibition Assay

The cytotoxic activity of IL-13 toxin or IL-4 toxin was tested as described (Puri, R. et al., Blood 87,4333-4339 (1996a); Puri, R. K. et al., Cancer Res. 56, 5631-5637 (1996b)). Typically, 10⁴ cells were cultured in leucine-free medium with or without various concentrations of IL13-PE38QQR or II4(38-37)-PE38KDEL for 20-22 hr at 37° C. For blocking experiments, cells were preincubated with IL13 or IL-4 (2 μg/ml) for 1 hr at 37° C. prior to the addition of IL-13 toxin to cells. Then 1 μCi of [³H]leucine (NEN Research Products, Boston, Mass.) was added to each well and incubated for an additional 4 hr. Cells were harvested and radioactivity incorporated into cells was measured with a beta plate counter (Wallac).

Clonogenic Assay

The in vitro cytotoxic activity of IL13-PE38QQR on A253 and PANC-1 cells (control cells or IL-1 3Rα-transfected cells) was also determined by colony-forming assay (Husain, S. R. et al., Clin. Cancer Res. 3, 151-156 (1997)). The cells were plated in triplicate in 100-cm² petri dishes with 7 ml of medium containing 20% PBS and were allowed to attach for 20-22 hr. The number of cells per plate was chosen such that more than 100 colonies were obtained in the control group. The cells were exposed to different concentrations of IL-13 toxin (0-100 ng/ml) for 14 days at 37° C. in a humidified incubator. The cells were washed, fixed, and stained with crystal violet (0.25% in 25% alcohol). Colonies consisting of more than 50 cells were scored. The percentage of surviving colonies was determined relative to the number of colonies formed in the control and treated groups.

B. Results of the Studies Reported in this Example

Cancer cells show increased binding to ¹²⁵I-labeled IL-13 after transfection with IL-13Rα chain.

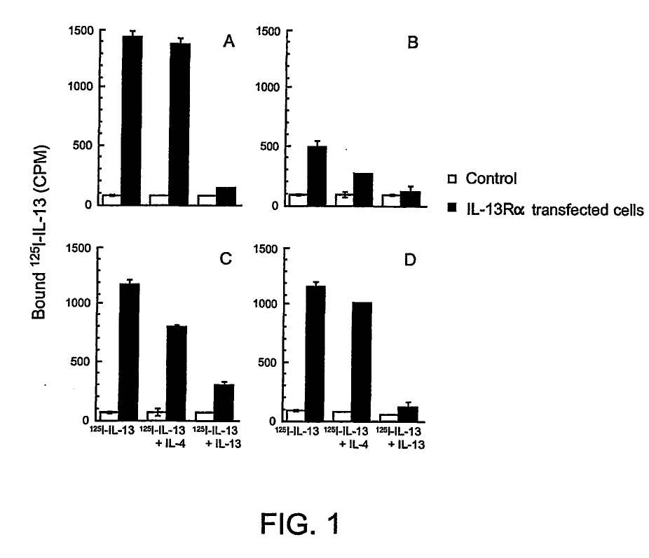

Four cancer cell lines from various pathological types, T98G, A253, Caki-1, and PANC-1, were shown to express no or low levels of IL-13Rα chain (Murata, T. et al., Cell. Immunol. 175, 33-40(1997). (Consequently, these cell lines show little binding to ¹²⁵IL-13 (FIG. 1). However, when these cells were transfected with IL-13Rα chain, binding activity of ¹²⁵IL-13 was dramatically increased. An excess of unlabeled IL-13 inhibited the binding of ¹²⁵IL-13, indicating specificity. Since IL-13R and IL-4R have been shown to share two chains with each other, it was examined whether IL-4 can displace IL-13 binding in these cells (Murata, T. et al., Blood 91, 3884-3891 (1998c)). Interestingly, in A253 and Caki-1 cell lines, IL-4 partially displaced ¹²⁵I-IL-13 binding; however, IL[-13 was superior to IL-4 in displacing ¹²⁵I-IL-13 binding. In the case of T98G and PANC-1 cell lines. IL-4 showed only a little displacement of ¹²⁵IL-13 binding. These findings indicate a difference in receptor structure and confirin previous results that IL-13R structure is different in different cell types (Obiri, N. I. et al., J. Immunol. 158, 75&764 (1997a)). These findings firer indicate that the receptors on T98G and PANC-1 cells behave like type I IL-13 receptors and those on Caki-1 and A253 cells behave like type II IL-13 receptors (Obiri, N. I. et al., J. Biol. Chem. 270, 8797-8804 (1995); Murata, T. et al., Int. J. Cancer 70, 230-240 (1997a); Murata, T. et al., Biochem. Biophys. Res. Commun. 238, 90-94 (1997b), Mirata, T. et al., Int. Immunol. 10. 1103-1110 (1998a); Murata, T. et al., Int. J. Mol. Med. 1, 551-557 (1998b)). From these experiments, the number of IL-13-binding sites pre- and posttransfection of IL-13Rα chain was calculated. As shown in Table 1, after transfection of IL-13Rα chain IL-13-binding sites increased 30- to 6000-fold compared with control cells.

TABLE 1
IL-13R-Binding Sites on Cancer Cell Lines and IL-13Rα2
Chain Transfectants and Cytotoxicity of IL-13 Toxin

| | IL-13R-binding sites (sites/cell) | | IC₅₀(ng/ml)^{b} | |
|---|---|---|---|---|
| | | IL-13Rα2 | | IL-13Rα2 |
| | Control^{a} | transfectants | Control^{a} | transfectants |
| Cell line | Mean ± SD | Mean ± SD | Mean ± SD | Mean ± SD |
| T98G | ND | 6000 ± 50 | >1000 | 0.7 |
| A253 | 20 ± 3.0 | 1600 ± 100 | 56 ± 4.0 | 5.0 ± 10.5 |
| Caki-1 | 140 ± 10 | 5300 ± 20 | 580 ± 30 | 95 ± 5.0 |
| PANC-1 | 160 ± 15 | 5100 ± 60 | 63 ± 4.0 | 2.8 ± 1.8 |

Abbreviations: ND, Not detectable; SD, standard deviation.
^{a}Cells transfected with vector only served as control.
^{b}IC50 the concentration of IL-13 toxin at which 50% inhibition of protein synthesis is observed compared with untreated cells.

Cancer Cells Transfected with IL-13Rα2 Chain Show Increased Sensitivity to IL13-PE38QQR

Protein synthesis inhibition assay. A chimeric protein composed of IL-13 and a truncated form of Pseudomonas exotoxin (IL-13-PE38QQR) was produced, which was found to be potently cytotoxic to IL-13R-positive solid tumor cells (Debinski W. et al., J. Biol. Chem. 270, 16775-16180 (1995a); Debinsid, W. et al., Clin. Cancer Res. 1, 1253-1258 (1995b); Puri, R. K. et al., Blood 87,4333-4339 (1996a); Husain, S. R. et al., Clin. Cancer Res. 3, 151-156 (1997); Maini, A. et al., J. Urol. 158, 948-953 (1997); Husain, S. R. et al., Blood 95, 3506-3513 (2000)). However, in cells that do not express or express very little IL-13R (especially IL-13Rα chain), IL-13 toxin is not very cytotoxic (Murata, T. et al., Int. J. Cancer 70, 230-240 (1997a)). Therefore, it was examined whether introduction of IL-13Rα chain can increase the sensitivity of these cells to IL-13 toxin. As shown in FIG. 2, transfection of IL-13Rα chain improved the sensitivity of all four cell lines to the cytotoxic effect of IL-13 toxin. The concentration of IL-13 toxin that causes 50% inhibition in protein synthesis (IC₅₀) in the four IL-13Rα transfected cancer cell lines improved from 6-fold to greater than 1000-fold compared with control cells (Table 1). The increase in sensitivity to IL-13 toxin correlated with the increase in IL-13R-binding sites. The cytotoxic activity of IL13-PE38QQR in IL-13Rα transfected cells was blocked by an excess of L-13 in all cell lines tested, indicating that cytotoxicity mediated by IL-13 toxin is specific.

It is of interest to note that although A253 cells express a lower number of IL-13R compared with Caki-1 cells; A253 cells were more sensitive to the cytotoxic effect of IL13-PE38QQR. The reason for this unexpected result is not known. Generally, the number of receptors correlates with the sensitivity to IL13-PE38QQR Rur, R. K. et al., Blood 87, 4333-4339 (1996); Puri, R. K. et al., Cancer Res. 56, 5631-5637 (1996)). It is possible that other IL-13 receptor components may influence the internalization rate in Caki-1 cells, making them less sensitive. Alternatively, PE may be less efficiently processed in the intracellular compartment in Caki-l cells as compared to A253 cells.

Similar to the binding data, IL-4did not block the cytotoxicity of IL-13 toxin in T98G cells and PANC-1 cells, while it did in Caki-1 and A253 cells. These results confirm that IL-13R on T98G and PANC-1 cells are distinct and do not interact with IL-4 (type I IL-13R). However, IL-13R in Caki-1 and A253 cells interact with IL-4 (type II IL-13R) (Obiri, N. I. et al., J. Biol. Chem. 270, 8797-8804 (1995)). It has previously been shown that IL-13R structure is different in different cell types (Obiri, N. I. et al., J. Immunol. 158, 756-764 (1997); Obiri, N. I. et al., J. Biol. Chem. 272, 20251-20258 (1997)). In some cell types, IL-4 cannot compete for the binding of radiolabeled IL-13 or block cytotoxicity mediated by IL-13-PE38QQR. This is because these cells express type I IL-13R in that IL-13-binding proteins (IL-13Rα′ and IL-13Rα) are coexpressed with primary IL-4-binding protein (IL-4Rβ, also known as IL-4Rα). With the introduced IL-13Rα chain, the IL-13R complex in transfected cells is now composed of IL-13Rα, IL-13Rα′, and IL-Rβ (type I IL-13R) chains. Because of this arrangement IL-13 or IL-13-PE38QQR binds to all three chains and IL-13 can compete for this binding. Because IL-4 binds to only two chains (IL-13Rα′ and IL-4Rβ) and IL-13 binds to IL-13Rα chain with high affinity and this chain is in excess, it does not allow competition by IL-4. However, in type II IL-13R, the a chain is absent and thus both IL-4 and IL-13 bind to both remaining chains of IL-4 and IL-13 receptors (IL-13Rα′ and IL4Rβ). Because of this arrangement both cytokines can displace the binding of IL-13 and IL-4 can reverse the cytotoxic effect of IL-13-PE38QQR. Thus, the receptors on Caki-1 and A253 cells behave like type II IL-13R even though these cell lines were tansfected with IL-13Rα chain. Therefore, it is possible that in these cell lines enough IL-13Rα was expressed to result in enhancement of sensitivity to IL-13-PE38QQR, but not enough was expressed to maintain high binding to IL-13-PE38QQR. This phenomenon has been previously observed in various cancer cell lines that were not transfected with any chain (Debinski, W. et al., J. Biol. Chem. 270, 16775-16180 (1995); Debinski, W. et al., Clin. Cancer Res. 1, 1253-1258 (1995); Obiri, N. I. et al., J. Immunol. 158, 756-764 (1997)). Studies are ongoing to unravel the exact molecular reasons for this diversity of interaction between IL-4 and IL-13 in different cell types.

To confirm that IL-13Rα chain does not interact with IL-4, the sensitivity of T98G IL-13Rα transfected cells to IL-4 toxin, 1L4(38-37)-PE38KDEL, was examined. It has previously been shown that human cancer cells that express IL-4R are very sensitive to the cytotoxic effect of IL-4 toxin (Kreitman, R. J. et al., Proc. Natl. Acad. Sci. U.S.A. 91, 6889-6893 (1994); Kreitman, R. J. et al., Cancer Res. 55, 3357-3363 (1995); Puri, R. K. et al., Cancer Res. 56, 5631-5637 (1996)). T98G cells express functional IL-4 receptors and IL-4 toxin is highly cytotoxic to these cells (Puri, R. K. et al., Int. J. Cancer 58, 574581 (1994), Pun, R. K. et al., Cancer Res. 56, 5631-5637(1996)). On transfection of IL-13Rα chain, sensitivity of these cells to IL-4 toxin was not increased (IC₅₀ of 2-3 ng/ml) (data not shown). These results confirm that IL-4R do not utilize IL-13Rα chain for internalization or signaling (Murata, T. et al., Blood 91, 3884-3891 (1998)).

Clonogenic assay. In vitro clonogenic assays were performed to examine the effect of IL13-PE38QQR on the proliferation of A253 and PANC-1 cells transfected with IL-13Rα chain. As shown in Table 2, although A253 cells and PANC-1 cells demonstrated some sensitivity to IL13-PE38QQR (IC₅₀ of 40 and 60 ng/ml, respectively), IL-13Rα-transfected cells showed five to nine times higher sensitivity (IC₅₀ of 7.5 and 6.7 ng/ml, respectively). The IC₅₀ values of IL13-PE38QQR by clonogenic assay corroborated well with the IC₅₀ values determined by protein synthesis inhibition assays.

TABLE 2
In Vitro Inhibition of PANC-1 and A253 Cell
Growth by IL-13 Toxin in a Clonogenic Assay

| | PANC-1 | | A253 | |
|---|---|---|---|---|
| IL13-PE38QQR | | IL-13Rα | | IL-13Rα |
| (ng/ml) | Control^{a} | transfectants | Control^{a} | transfectants |
| 0.1 | 95 ± 4^{b} | 100 ± 3 | 100 ± 4 | 100 ± 4 |
| 1 | 82 ± 5 | 76 ± 8 | 98 ± 4 | 87 ± 4 |
| 5 | 82 ± 3 | 64 ± 4 | 100 ± 7 | 75 ± 2 |
| 10 | 66 ± 4 | 28 ± 4 | 81 ± 4 | 37 ± 2 |
| 100 | 43 ± 1 | 11 ± 2 | 19 ± 4 | 2 ± 1 |
| IC₅₀(ng/ml): | 60 | 6.7 | 40 | 7.5 |

^{a}Cells transfected with vector alone served as control.
^{b}Results are expressed as percentage of colonies formed by treated cells compared with untreated cells. PANC-1 (control), PANC-1 (IL-13Rα transfectants), A253 (control), and A253 (IL-13Rα transfectants) cells formed 214 ± 6, 91 ± 3, 123 ± 4, and 108 ± 6 colonies (mean ± SD), respectively. The IC₅₀ by clonogenic assay was calculated from the results.

C. Discussion of the Results of the Studies Reported in this Example

Four cancer cell lines which express no or low numbers of IL-13R were shown to bind IL-13 at much higher levels after transfection of IL-13Rα chain. IL-13Rα-transfected cells become highly sensitive to IL13-PE38QQR compared with control cells. Because clonogenicity in vitro correlates with in vivo malignant phenotype in xenografts Freedman, V. H. et al., Cell 3, 355-359 (1974); Gross, S, et al., Cancer Res. 48, 291-296 (1988)), the data also suggest that antitumor activity of IL13-PE38QQR.

This is the first demonstration that cancer cells that do not express IL-13R or demonstrate low or no sensitivity to IL-13R-targeted cytotoxins can change their sensitivities dramatically after genetic transfer of only one chain of a cytokine receptor. IL13-PE38QQR was found to be cytotoxic only to cancer cells and not to human T and B cells, monocytes, normal endothelial cells, and resting or growth factor-activated bone marrow cells (Puri, R. K. et al., Blood 87,4333-4339 (1996)). Furthermore, it has been observed in this and previous studies that there was a positive correlation between the level of IL-13R expression and sensitivity to the IL13-PE38QQR (Debinski, W. et al., J. Biol. Chem. 270, 16775-16180 (1995); Debinsld, W. et al., Clin. Cancer Res. 1, 1253-1258 (1995); Puri, R. K. et al., Blood 87, 4333-4339 (1996); Husain, S. R. et al., Clin. Cancer Res. 3, 151-156 (1997)). Taken together, the new findings offer outstanding possibilities for the utilization of IL-13 toxin for cancer therapy.

This new strategy, which introduces a functional cytokine receptor chain into cancer cells, offers a novel and widely useful technique for immunotherapy.

### Example 2

This Example reports in vivo studies regarding the sensitization of cancer cells by transfection with the IL-13Rα2 chain, and the regression of tumors of such cells upon administration of anti-IL-13R immunoconjugates either systemically or intratumorally.

A. Materials and Methods Used in the Studies Reported in this Example

Recombinant Cytokines and Toxin. Recombinant human IL-4 and IL-13 were produced and purified to homogeneity in the laboratory. Recombinant IL13-PE38QQR was also produced and purified in our laboratory (Debinski, W. et al., J. Biol. Chem., 270:16775-16780 (1995).

Cell Lines. Human head and neck cancer cell lines (SCC-25 and A253) were purchased from the American Type Culture Collection (Manassas, Va.). KCCTC873 (termed KCCT873), YCUT891, and KCCT871 cell lines were established in the Department of Otolaryngology, Yokohama City University School of Medicine or Research Institute, Kanagawa Cancer Center (Yokohama, Japan). Cells were cultured in DMEM-Ham's F12 (SCC-25), McCoy's 5A (A253) or RPMI1640 (the other cell lines) containing 10% fetal bovine serum (Biowhittaker Inc., Walkersville, Md.), 1 mM HEPES, 1 mM L-glutamine, 100 μg/ml penicillin, 100 μg/ml streptomycin (Biowhittaker Inc.), and 400 ng/ml hydrocortisone (only for SCC-25; Sigma Chemical Co., St. Louis, Mo.).

Stable Transfection and Selection. cDNA encoding human IL-13Rα2 chain (Caput, D. et al., J. Biol. Chem., 271:16921-16926 (1996)) was cloned into pME18S mammalian expression vector (Murata, T. et al., Blood, 91: 3884-3891 (1.998)). Plasmid DNA (12 μg/100-mm culture dish) was co-transfected with 1.2 μg of pPUR selection vector (Clontech Laboratories, Inc. Palo Alto, Calif.) into semiconfluent cells using GenePORTER transfection reagent (Gene Therapy Systems, San Diego, Calif.) according to the manufacturer's instructions. Briefly, cells (2×10⁶/100-mm dish) were incubated with the DNA-GenePORTER mixture for 5 h in DMEM (Biowhittaker). Then DMEM containing 20% FBS was added and incubation was continued. Twenty four hours after transfection, the medium was changed to DMEM with 10% FBS and cells were incubated for an additional 24 h. At 48 h after the start of transfection, cells were trypsinized and cultured in selection medium that contained 1 μg/ml of puromycin (Clontech Laboratories, Inc.). Cells were maintained for 4 weeks in the same medium, which was replaced every 3 days. Resistant clones (twenty-five A253 clones, thirteen YCUT891 clones, and five KCCT871 clones) isolated with the cloning cylinder (Bel-Art products, Pequannock, N.J.) were characterized for IL-13Rα2 chain expression by RT-PCR and radioreceptor binding assays. Finally, one each IL-13Rα2-overexpressing clones (termed A253α2, YCUT8912, and KCCT871α2) were selected for further analysis. The vector control (mock) transfected cell lines A253mc, YCUT891mc, and KCCT871mc were used for comparison with IL-13Rα2 transfected cells. To reduce antibiotic side effects, puromycin was removed at least 14 days before experiments were performed.

RT-PCR Analysis. To detect the mRNA expression of IL-13R chains in SCCHN cells, total RNA was isolated using TRIZOL reagent (Life Technologies, Grand Island, N.Y.), then RT-PCR analysis was performed. Two jig of total RNA was incubated for 30 min at 42° C. in 20 μl reaction buffer containing 10 mM Tris-HCl (pH 8.3), 5 mM MgCl₂, 50 mM KCl, 1 mM each of dNTPs, 1 unit/μl RNase inhibitor, 2.5 μM random hexamer, and 2.5 units/μl of MMLV RT (Perldn-Elmer Corp., Norwalk, Conn.). A 10-μl aliquot of RT reaction was amplified in 100-μl final volume of PCR mixture containing 10 mM Tris-HCl (pH 8.3), 2 mM MgCl₂, 50 mM KCl, 1 unit of AmpliTaq Gold DNA polymerase (Perkin-Elmer Corp.), and 0.1 μg of specific primers for IL-13Rα2, IL-13Rα1, IL-4Rα, or γ_{c} chains (Murata, T. et al., Biochem. Biophys. Res. Commun., 238:9094 (1997)). PCR product (30 μl) was run on a 2% agarose gel for UV analysis.

Radioreceptor Binding Assays. Recombinant human IL-13 or IL-4 were labeled with ¹²⁵I (Amersham Corp., Arlington Heights, Ill.) using IODO-GEN reagent (Pierce, Rockford, Ill.) as previously described (Obiri, N. I. et al., J. Clin. Invest., 91:88-93 (1993)). The specific activity of the radiolabeled cytokines were estimated to be 6.0 μCi/μg (IL-13) or 28 μCi/μg (IL-4) of protein. For binding experiments, 5×10⁵ cells in 100 μl binding buffer [RPMI 1640 containing 0.2% human serum albumin (HSA) and 10 mM HEPES] were incubated with 200 μM ¹²⁵I-IL-13 or ¹²⁵I-IL-4 with or without 40 nM unlabeled IL-4 or IL-13 at 4° C. for 2 h. Cell-bound radiolabeled cytokine was separated from unbound by centrifugation through a phthalate oil gradient and radioactivity was determined with a gamma counter (Wallac, Gaithersburg, Md.). Binding sites/cell were calculated based on the specific binding of radiolabeled cytokine as previously described (Kawakami, K. et al., Hum. Gene Ther., 11:1829-1835 (2000)).

Protein Synthesis Inhibition Assay. The cytotoxic activity of IL-13 toxin was tested as previously described (Puri, R. K. et al., Cancer Res., 51:3011-3017 (1991)). Typically, 10⁴ cells were cultured in leucine-free medium with or without various concentrations of IL13-PE38QQR for 20-22 h at 37° C. Then 1 μCi of [³H]leucine (NEN Research Products, Boston, Mass.) was added to each well and incubated for an additional 4 h. Cells were harvested and radioactivity incorporated into cells was measured by a β plate counter (Wallac).

Animals. Athymic nude mice 4 weeks old (about 20 g in body weight) were obtained from Frederick Cancer Center Animal Facilities (National Cancer Institute, Frederick, Md.). The mice were housed in filter-top cages in a laminar flow hood in pathogen-free conditions with 12 h light/12 h dark cycles. Animal care was in accordance with the guidelines of the NIH Animal Research Advisory Committee.

Human Head and Neck Cancer Xenografts and Treatment Human head and neck tumors were established in nude mice by subcutaneous injection into the flank of 5×10⁶ SCC-25, KCCT873, A253mc, A253α2, YCUT891mc, or YCUT891α2 cells in 150 μl of PBS plus 0.2% HAS (cells listed above that end with the “α2” designation denote that cells of that cell line transfected with the IL-13Rα2 chain). Palpable tumors developed within 3-4 days. The mice then received injections of excipient (0.2% HSA in PBS) or chimeric toxin either intraperitoneally (“IP;” 500 μl) or intratumorally (“IT;” 30 μl) using a 22-gauge needle.

Statistical Analysis. Tumor sizes were calculated by multiplying length and width of tumor on a given day. The statistical significance of tumor regression was calculated by Student t test.

B. Results of the Studies Reported in this Example

Subunit Structure of IL-13R on Head and Neck Cancer Cells. Five SCCHN cell lines were examined for the expression of mRNA for various putative IL-13R subunits (IL-13Rα2, IL-13Rα1, IL-4Rα, and γ_{c} chains) by RT-PCR. In each case, 2 μg of total RNA was examined. mRNA for IL-13Rα1 and IL-4Rα chains were present in all of the cell lines examined. However, no SCCHN cell lines showed presence of γ_{c} mRNA. Very low level or no expression of IL-13Rα2 chain was observed in A253mc, YCUT891mc, and KCCT871mc cells. As expected, IL-13Rα2-transfected cell lines (A253α2, YCUT891α2, and KCCT871α2) showed ample mRNA expression. PM-RCC cells that express IL-13Rα2, IL-13Rα1, and IL-4Rα chains and H9 T lymphoma cells that express γ_{c} mRNA served as positive controls.

IL-13 Binding to IL13Rα2 Chain-Positive and -Negative SCCHN Cell Lines. The expression and binding affinity of IL-13R on SCCHN cell lines was determined by ¹²⁵I-IL-13 binding assays. Two IL-13Rα2 chain-positive cell lines and three negative cell lines and transfectants were labeled with ¹²⁵I-IL-13 in the absense or presence of 200-fold molar excess of IL-13. Cells (5×10⁵) were incubated at 4° C. for 2 hours with 200 pM ¹²⁵I-IL-13. ¹²⁵I-IL-13 bound to SCCHN cells at almost same degree and an excess of unlabeled IL-13 displaced the binding of ¹²⁵I-IL-13. IL-13R and IL-4R share two chains, therefore, a study was conducted to determine whether IL-4 can also displace the IL-13 binding in SCCHN cells (Murata, T. et al., Int. J. Mol. Med., 1:551-557 (1998); Kawakami, K. et al., Cancer Res., 60:2981-2987 (2000); Murata, T. et al., Blood, 91: 3884-3891 (1998)). Cells were incubated as described above with or without 40 nM of unlabeled IL-4 or IL-13. The findings indicated that IL-4 also displaced ¹²⁵I-IL-13 binding in KCCT873 cells; however, in SCC-25 cells, IL-4 showed only minimal displacement of ¹²⁵I-IL-13 binding.

The three SCCHN cell lines which have no IL-13 binding component, IL-13Rα2 chain, showed little binding to ¹²⁵I-IL-13. However, when these cells were transfected with IL-13Rα2 chain, the binding activity of ¹²⁵I-IL-13 was dramatically increased. An excess of unlabeled IL-13 inhibited the binding of ¹²⁵I-IL-13, indicating specificity. Interestingly, unlabeled IL-4 showed minimal displacement of ¹²⁵I-IL-13 binding in YCUT891 and KCCT871 cell lines. On the other hand, IL-4 partially displaced ¹²⁵I-IL-13 binding in A253 cell line. As SCCHN cell lines express IL-4R, IL-4 binding sites in these cells were also determined (Kawakami, K et al., Cancer Res., 60:2981-2987 (2000)). From these experiments, the number of IL-13-binding sites on IL-13Rα2 chain-positive and negative cell lines was calculated. As shown in Table 3, in IL-13Rα2 chain-negative cell lines, after transfection of IL-13Rα2 chain IL-13-binding sites increased 48- to 850-fold compared with control cells. However, IL-4 binding sites did not increase in IL-13Rα2-transfected cells except in A253 cells that showed slight increase in the number of IL-4 binding sites.

SCCHN Cells Transfected with IL-13Rα2 Chain Show Increased Sensitivity to IL13-PE38QQR. A chimeric protein composed of IL-13 and a truncated form of Pseudomonas exotoxin (IL13-PE38QQR), which was found to be potently cytotoxic to IL-13R-positive solid tumor cells, has been previously reported (Debinski, W. et al., J. Biol. Chem., 270:16775-16780 (1995); Puri, R. K. et al., Blood, 87:43334339 (1996); Husain, S. R. et al., Clin. Cancer Res., 3:151-156 (1997); Maini, A. et al., J. Urol., 158:948-953 (1997), Husain, S. R. et al., Blood, 95:35063513 (2000)). To determine whether IL-13Rα2 chain-positive SCCHN cell lines are sensitive to IL-13 immunoconjugates, this molecule was selected as an exemplary cytotoxin and its cytotoxicity tested against SCC-25 and KCCT873 cells. IL-13 toxin was cytotoxic to these cell lines, and the IC₅₀ (the protein concentration required for the inhibition of protein synthesis by 50%) were 2.4 and 4.0 ng/ml, respectively (Table 1). The cytotoxic activity of IL13-PE38QQR was neutralized by excess IL-13 and partially by IL-4 only in the KCCT873 cell line.

In cells that do not express or express very little IL-13Rα2 chain, IL-13 toxin is minimally cytotoxic. Therefore, to explore whether introduction of this chain into the cells increase the sensitivity of IL-13 toxin, stable transfectants of this chain were used. Transfection of IL-13Rα2 chain improved the sensitivity of all three cell lines to the cytotoxic effect of IL-13 toxin. IC₅₀s in the three cell lines improved from 520-fold to 1000-fold compared with control cells. The increase in sensitivity to IL-13 toxin correlated with the increase in IL-13R-binding sites. The cytotoxic activity of IL-13 toxin in IL-13Rα2-transfected cells was blocked by an excess of IL-13 in all three cell lines, indicating that cytotoxicity mediated by this molecule is specific. Similar to binding data, IL-4 partially inhibited the cytotoxic activity of IL-13 toxin in the A253α2 cell line.

Intraperitoneal Antitumor Activity of IL-13 toxin to IL-13Rα2 Chain-Positive SCCHN Tumors. To explore IL-13 toxin mediated antitumor activity to IL-13Rα2 chain-positive SCCHN cell lines, nude mice were implanted subcutaneously with 5×10⁶ SCC-25 or KCCT873 tumor cells on day 0. The mice were then injected intraperitoneally with IL13-PE38QQR (4 mice, treated with 50 μg/kg) or excipient only (5 mice, as controls) twice daily for 5 days from days 4 to 8 (a total of 10 injections). As shown in FIG. 3A, all SCC-25 tumors started regressing during the treatment and one tumor completely disappeared by day 8. Although one tumor began to appear on day 11, by day 43 the mean size of the tumors remained small similar to the size of tumors on the day of the first injection (23 mm²). By day 75, treated tumors gradually grew to 35 mm², and the reduction in tumor size was 74% (P<0.001) compared with control tumors (137 mm²)

As shown in FIG. 3B, in the KCCT873 tumor model, all tumors started regressing during the treatment and by day 8 tumors decreased to very small masses (7 mm²). Thereafter, the tumors started growing gradually; however, the size remained significantly smaller compared with control tumors. As tumors in control mice injected with vehicle only continued to grow exponentially, these mice were killed on day 36. The reduction in tumor size in treated group on day 36 was 75% (46 mm²; p<0.0006) compared with tumors in control group (180 mm²).

Intratumoral IL-13 toxin Treatment Induced Total Eradication of IL-13Rα2 Chain-Positive SCCHN Tumors. The efficacy of intratumoral administration of IL-13 toxin against SCC-25 and KCCT873 tumors was also assessed. Nude mice with established SCC-25 or KCCCT873 tumors received intratiumoral EL13-PE38QQR or excipient, as a control. Each injection was 250 μg/kg per day, injections were administered on days 4, 6, and 8. There were 5 mice in the control group (which received excipient only) and 4 in the treated group. The injected volume was 30 μl in each tumor. In the mice with SCC-25 tumors, the IL13-PE38QQR injections inhibited tumor growth and two of four tumors completely regressed by day 7 (see FIG. 4A). By day 11, the growth of all treated tumors which was arrested subsequently disappeared completely. Although a palpable tumor appeared in one mouse on day 15, three mice remained tumor-free until the day they were killed (day. 90).

Treatment of KCCT873 tumors with intratumoral IL-13 toxin (250 μg/kg per day on alternate days for 3 days) reduced the tumor size and one of four tumors showed complete regression by day 7 (see FIG. 4B). By day 11, one more tumor disappeared in the treated mice group. On day 15, the palpable tumors appeared in those mice and all the tumors began to grow gradually; however, the size of the tumor was significantly smaller and the reduction in tumor size in treated group on day 36 was 77% (41 mm²; P<0.0008) compared with tumors in the control group injected with vehicle-only (180 mm²).

Sensitivity of IL-13Rα2 Chain-Negative SCCHFN Tumors to Intraperitoneal Administration of IL-13 Toxin Was Dramatically Increased by IL-13Rα2 Chain Gene Transfer. Transfection of IL-13Rα2 chain was found to improve dramatically the sensitivity of SCCHN cell lines to the cytotoxic effect of IL-13 toxin in vitro. To determine whether these results also obtained in vivo, nude mice were implanted subcutaneously on day 0 with either 5×10⁶ A253 or YCUT891mc cells transfected only with the vector, as a control or with IL-13Rα2 chain transfected cells A253α2 or YCUT891α2. Each group had 5 animals. The animals then received twice a day IP injections (50 μg/kg) with IL13-PE38QQR or with excipient only (as a control). YCUT891mc and YCUT891α2 tumor bearing mice received a second course of injections with the same doses on days 25 to 29 post implantation.

As shown in FIG. 5A, in A253mc tumor-bearing mice, the tumors grew very well and the tumors treated with IL-13 toxin (50 pg/kg) twice daily for 5 days (total 10 injections) did not result in significant reduction in size. On day 52, both treated mice and vehicle-only injected mice were sacrificed and the size was 190 mm² and 168 mm², respectively.

On the other hand, as shown in FIG. 5B, in nude mice bearing A253 tumors transfected with IL-13Rα2 chain (A253α2 tumors), the tumors grew as fast as vector-only transfected A253mc tumors, but IL-13 toxin (50 μg/kg) treatment in the same schedule as in the A253mc group (twice daily for 5 days) resulted in significant antitumor activity. Two of five mice showed complete disappearance of their tumors by 4 days after the first injection. By day 24, two more tumors showed a complete regression. These mice remained tumor-free until day 52. Only one mouse had even a very small tumor. On day 52, the reduction in tumor size in treated group was 95% (10 mm²; P<0.00002) compared with tumors in vehicle only injected control group (187 mm²).

In a further set of studies, the results of which are shown in Figure FIG. 5C, YCUT891-tumor bearing mice (tumors lacking high levels of IL-13α2 chain) were also injected with IL13-PE38QQR (50 μg/kg) twice daily for 5 days from day 4 to 8. In addition, these mice also received a second course on day 25 through 29. YCUT891mc tumors showed no sensitivity to IL-13 toxin upon intraperitoneal administration even after the second course of the treatment. In contrast, as shown in FIG. 5D, after the first course treatment with IL-13 toxin (50 μg/kg) from day 4 to 8, YCUT891α2 tumors began to regress gradually. While no tumor disappeared completely, the tumors remained smaller in size (about 24 mm²) compared to untreated mice. However, when mice were given the second course of IL-13 toxin (50 μg/kg) treatment from day 25 to 29, the tumors began to regress again. By day 56, all the tumor size remained small similar to size on the day of injection (33 mm²) and the reduction in tumor size in treated group was 80% (41 mm²; P<0.0001) compared with tumors in vehicle only injected control group (210 mm²).

Complete Regression of IL-13Rα Chain-transfected SCCHN Tumors with IL-13 Toxin Intratumoral Treatment. To assess the efficacy of intratumoral treatment with IL-13 toxin to IL-13Rα2 chain-negative SCCHN tumors, IL-13Rα2 transfected tumors, nude mice with established A253α2 or YCUT891α2 tumors were intratumorally treated with IL13-PE38QQR or with excipient only, as a control. Each group had 5 animals.

A253α2 tumors were treated with IL-13 toxin or excipient, as a control (250 μg/kg per day on alternate days for 3 days from day 4). As shown in FIG. 6A, by day 7 tumors in two of five mice treated with the IL-13 toxin disappeared completely. By day 24, 100% of the tumors were completely regressed. All treated mice remained tumor-free until day 52, when the experiment was terminated. By contrast, mice treated with excipient only exhibited robust tumor growth.

Mice with YCUT891α2 lot tumors were intratumorally treated for two courses with IL-13 toxin (250 μg/kg per day on alternate days for 3 days; the injected volume was 30 μl in each tumor) from days 4 to 8 and from days 25 to 29. The results are shown in FIG. 6B. After the first treatment course, tumors began to decrease in size, however, from day 14 tumors started growing again. No complete responders were observed at that time. After the second course of IL-13 toxin therapy, tumors began to regress again, and by day 28 three of five tumors completely disappeared. By day 49, two mice developed recurrence, however, one mouse remained tumor free until day 56. The reduction in tumor size in treated group on day 56 was 86% (29 nm2; p<0.00003) compared with tumors in vehicle only injected control group (210 mm²)

C. Discussion of the Results Reported in this Example

These studies demonstrate that not only IL-13Rα2 chain-positive head and neck cancer cell lines but also IL-13Rα2 chain-negative cell lines can be dramatically sensitized to the antitumor activity of IL-13 toxin after gene transfer for IL-13Rα2 chain. SCCHN cell lines were classified by the presence or absence of the IL-13Rα2 chain. Although RT-PCR analysis did not directly confirm the expression of IL-13R chains, the studies imply that the IL-13R complex in SCCHN cell lines represents type I (where the IL-13Rα1 and IL-13Rα2 chains co-exist on the cell surface) or type II (where the IL-13Rα1 and IL-4Rα chains form a complex) IL-13R. The common y, chains was not identified in these cells. The reason why some SCCHN cell lines express IL-13Rα2 chain is not known. In 17 different SCCHN cell lines, only 20% cell lines were found to express IL13Rα2 chain. The significance of over-expression of IL13Rα2 chain is currently being investigated.

Interestingly, in KCCT873 cells, IL-4 was able to displace ¹²⁵I-IL-13 binding while in SCC-25 cells IL-4 did not. Furthermore, in A253 cells transfected with IL-13Rα2 chain, IL-4 was able to displace ¹²⁵I-IL-13 while in YCUT891α2 and KCCT871α2 cells IL-4 did not. These results are consistent with previous studies that have demonstrated that IL-4 can compete for the ¹²⁵I-IL-13 binding sites on some cell lines while not on others (Obiri, N. I. et al., J. Biol. Chem., 270:8797-8804 (1995); Murata, T. et al., Int. J. Cancer, 70:230-240 (1997); Obiri, N. I. et al., J. Immunol., 158:756-764 (1997); Obiri, N. I. et al., J. Biol. Chem., 272:20251-20258 (1997); Murata, T. et al., Int. J. Mot. Med., 1:551-557 (1998); Hilton, D. J. et al., Proc. Natl. Acad. Sci. U S. A., 93:497-501 (1996); Caput, D. et al., J. Biol. Chem., 271:16921-16926 (1996)). This interesting phenomenon may be explained by the stoichiometry of different receptor chain expression. If cells constitutively express high levels of IL4Rα chain, IL-4 will be able to displace both ¹²⁵I-IL-13 binding and ¹²⁵-IL-4 binding. If the level of expression of this chain is lower, then IL-4 does not displace ¹²⁵I-IL-13 binding. The ¹²⁵I-IL-4 binding studies conducted in the present work partly support this conclusion. But in SCC-25 cells that expressed higher binding sites (13,000) than KCCT873 (7600), IL-4 did not displace for ¹²⁵I-IL13 binding. These results suggest that alternative mechanism(s) may also exist for this complex interaction between IL-4R and IL-13R.

Both IL-13Rα2-positive and IL-13Rα2 stably transfected SCCHN cell lines showed high sensitivity to IL-13 toxin as assessed by cytotoxicity assays. However, SCCHN cells that did not express this chain were not sensitive. These data suggest that the IL-13Rα2 chain is necessary for internalization of sufficient molecules of immunotoxin for cytotoxicity to inhibit cell growth. Investigation of the mechanism of cell death indicated that 30-46% of SCCHNcells died through apoptotic cell death by IL13-PE38QQR while IL-13 alone had no effect.

Consistent with in vitro sensitivity results, IL-13 toxin showed pronounced antitumor activity in vivo against tumors that expressed IL-13Rα2 chain naturally or artificially. In two animal models, IL-13 toxin showed very high antitumor activity; however, when IL-13 toxin was administrated IP, no complete responders were observed. Upon intratumoral administration, IL-13-PE produced complete responders in the SCC-25 tumor model but not in the KCCT873 tumor model. On the other hand, IL-13Rα2 chain-negative tumors (A253mc and YCUT891mc) did not respond to IL-13 toxin at all by either the IP route or the IT route even with two courses of IL-13 toxin. However, when IL-13Rα2 chain-transfected tumor (A253α2)-bearing mice were injected with IL13-PE38QQR interperitoneally, 4 of 5 mice showed complete disappearance of disease. Similarly, when the toxin was injected IT, all animals showed complete regression of tumors. Interestingly, when IL13Rα2 chain transfected YCUT891 tumor (YCUT891α2) bearing mice were injected with two courses of IL-13 toxin by either the IP or IT route, none of the animals showed a complete response. However, administration by either route showed a remarkable antitumor activity. The mechanism of lack of a complete response in IL-13Rα2 chain-transfected YCUT891α2 tumors is not known. It is possible that IL-13Rα2 chain gene expression was not optimal. Although YCUT891x2 tumor cells expressed IL-13Rα2 chain mRNA, quantitative comparisons of IL-13α2 chain expression could not be performed. Both A253α2 and YCUT891α2 cell lines expressed similar density of IL-13R (Table 1). Thus other mechanisms are operational in differential sensitivity to the IL-13 toxin in two tumor models. The efficiency of distribution of IL-13 toxin in the tumor bed may be another part of this difference.

This is the first demonstration that SCCHN cells that do not express IL-13R or have no or less sensitivity to IL-13R-targeted cytotoxins can change their sensitivities dramatically in vitro and in vivo after genetic transfer of only one chain of cytokine receptor. Because IL13-PE38QQR was found to be cytotoxic only to cancer cells that express IL-13R and not to human T and B cells, monocytes, normal endothelial cells, and resting or growth factor-activated bone marrow cells (Husain, S. R et al., Clin. Cancer Res., 3:151-156 (1997)), the current findings offer promising possibilities for the utilization of IL-13 toxin for both IL-13Rα2 chain-positive and -negative SCCHN cancer therapy.

Although various strategies are being developed for immunotherapy or targeting of cancer, this strategy is the only unique method that utilizes one cytokine receptor chain as a sensitizer to targeted cancer therapy.

Table 3 ZL-4R and IL-13R-Binding Sites on Head and Neck Cancer Cell Lines and Cytotoxicity ofIL-13 Toxin

For data of radioreceptor binding assays and protein synthesis inhibition assays, the number of IL-4 and IL-13-binding sites and cytotoxicity of IL-13 toxin to these cell lines were calculated.

| | IL-4R | IL-13R- | |
|---|---|---|---|
| | binding | binding | |
| | sites | sites | IC₅₀ |
| Cell line | (sites/cell) | (sites/cell) | (ng/ml)* |
| SCC-25 | 13000 ± 510 | 7800 ± 1200 | 2.4 ± 0.6 |
| KCCT873 | 7600 ± 810 | 5000 ± 290 | 4.0 ± 0.5 |
| A253mc | 6100 ± 650 | 190 ± 40 | 200 ± 50 |
| A253α 2 | 13000 ± 2800 | 13000 ± 200 | 0.2 ± 0.4 |
| YCUT891mc | 6300 ± 1200 | 20 ± 9.0 | 520 ± 80 |
| YCUT891α2 | 7100 ± 580 | 17000 ± 720 | <0.1 |
| KCCT871mc | 9100 ± 490 | 230 ± 60 | 300 ± 85 |
| KCCT871α2 | 8600 ± 60 | 11000 ± 570 | 0.3 ± 0.1 |

*IC₅₀, the concentration of IL-13 toxin at which 50% inhibition of protein synthesis is observed compared with untreated cells.

### Example 3

This Example shows the dramatic enhancement of sensitivity of prostate cancer cells to IL-13-targeted immunotoxins when the cells are transfected with IL-13Rα2 chain.

A. Materials and Methods Used in the Studies Reported in this Example

Recombinant Cytokines and Toxin

Recombinant human IL-4 and IL-13 were produced and purified as described (Oshima et al., J. Biol Chem., 275:14375-14380 (2000)). Recombinant IL13-PE38QQR in which IL-13 was fused to domain II and III of Pseudomonas exotoxin was also produced and purified.

Cell Lines and Culture

Human prostate cancer cell lines (DU145 and LNCaP) were purchased from the American Type Culture Collection (Rockville, Md.). Primary normal prostate cell lines (568NPTX and 570NP2TX) and prostate cancer cell lines (527CP2TX, 568CP1TX, and 570CP2TX) were established in Franz Cancer Research Center, Chiles Research Institute (Portland, Oreg.) (13right et al., Cancer Res., 57:995-1002 (1997)). Cells were cultured in Eagle's Modified Essential Medium (DU145) or RPMI1640 (LNCaP) containing 10% fetal bovine serum (Biowhittaker Inc., Walkersville, Md.), 1 mM HEPES, 1 mM L-glutamine, penicillin (100 μg/mL), and streptomycin (100 μg/mL) (Biowhittaker Inc.). The other primary cell lines were cultured in keratinocyte serum-free medium (Keratinocyte-SFM, Life Technologies Inc., Rockville, Md.) containing bovine pituitary extract (25 μg/mL), epidermal growth factor (5 ng/mL), 2 L-glutamine, 10 mM HEPES, antibiotics, and 5% fetal bovine serum.

Transfection and Selection

cDNA encoding human IL-13Rα2 chain (Caput et al., J. Biol Chem., 271:16921-16926 (1996)) was cloned into pME18S mammalian expression vector (Murata et al., Blood, 91:38843891.(1998)). Plasmid DNA (12 μg/100-mm culture dish) was transfected with or without 1.2 μg of pPUR selection vector (Clontech Laboratories, Inc., Palo Alto, Calif.) into semiconfluent cells using GenePORTER™ transfection reagent (Gene Therapy Systems, San Diego, Calif.) according to the manufacturer's instructions. Briefly, cells (2×10⁶/100-mm dish) were incubated with the DNA-GenePORTER™ mixture for 5 hours in DMEM (Biowhittaker). Then DMEM containing 20% FBS was added and incubation was continued. Twenty four hours after transfection, the medium was changed to DMEM with 10% FBS and cells were incubated for an additional 24 hours. About 48 hours after the start of transfection, cells were trypsinized and experiments were performed. For stable transfection, DU145 cells were further cultured in selection medium that contained 1 μg/mL of puromycin (Clontech Laboratories, Inc.). Cells were maintained for 4 weeks in the same medium, which was replaced every 3 days. Resistant clones isolated with the cloning cylinder (Bel-Art products, Pequannock, N.J.) were characterized for IL-13Rα2 chain expression by RT-PCR and radioreceptor binding assays. Finally, IL-13Rα2-overexpressing clone (termed DU145α2) was selected for further analysis. The vector control transfected (mock) cell line, termed DU145mc, was used for comparison with IL-13Rα2 transfected cells. To reduce antibiotic side effects on cell behavior, puromycin was removed at least 14 days before experiments were performed.

RT-PCR Analysis

To detect the mRNA expression of IL-13R chains in normal prostate and prostate cancer cells, total RNA was isolated using TRIZOL reagent (Life Technologies, Grand Island, N.Y.), then RT-PCR analysis was performed. Two μg of total RNA was incubated for 30 min at 42° C. in 20 μL reaction buffer containing 10 mM Tris-HCl (pH 8.3), 5 mM MgCl₂, 50 mM KCl, 1 mM each of dNTPs, 1 unit/μL RNase inhibitor, 2.5 μM random hexamer, and 2.5 units/μL of MMLV RT (Perkin-Ehmer Corp., Norwalk, Conn.). A 10-μL aliquot of RT reaction was amplified in 100-μL final volume of PCR mixture containing 10 mM Tris-HCl (pH 8.3), 2 mM MgCl₂, 50 mM KCl, 1 unit of AmpliTaq Gold DNA polymerase (Perkin-Elmer Corp.), and 0.1 μg of specific primers for either IL-13Rα2 or IL-13Rα1 chains (Murata et al., Biochem Biophys Res Commun., 238:90-94 (1997)). PCR product (30 μL) was run on a 2% agarose gel for UV analysis.

Radioreceptor Binding

Recombinant human IL-13 was labeled with ¹²⁵I (Amersham Corp., Arlington Heights, Ill.) using IODO-GEN® reagent (Pierce, Rockford, Ill.) as previously described (Obiri et al., J. Clin Invest., 91:88-93 (1993)). The specific activity of the radiolabeled cytokines were estimated to be 6.0 μCi/μg of protein. For binding experiments, 5×10⁵ cells in 100 μL binding buffer (RPMI 1640 containing 0.2% human serum albumin and 10 mM HEPES) were incubated with 200 pM ¹²⁵I-IL-13 with or without various concentrations (10 pM to 100 nM) of unlabeled IL-4 or IL-13 at 4° C. for 2 hours. Cell-bound ¹²⁵I-IL-13 was separated from unbound by centrifigation through a phthalate oil gradient and radioactivity was determined with a gamma counter (Wallac, Gaithersburg, Md.). In some experiments, the number of IL-13Rs and binding affinities were calculated using the LIGAND program (Munson and Rodbard, Anal Biochem 107:220-239 (1980)).

Protein Synthesis Inhibition Assay

The cytotoxic activity of IL-13 toxinwas tested as previously described (Puri et al., Cancer Res., 51:3011-3017 (1991)). Typically, 10⁴ cells were cultured in leucine-free medium with or without various concentrations of IL13-PE38QQR for 20-22 hours at 37° C. Then 1 μCi of [³H]leucine (NEN Research Products, Boston, Mass.) was added to each well and incubated for an additional 4 hours. Cells were harvested and radioactivity incorporated into cells was measured by a β plate counter (Wallac). The concentration of IL-13 toxin at which 50% inhibition of protein synthesis (IC₅₀) occurred was calculated.

Animals

Athymic nude mice 4 weeks old (about 20 g in body weight) were obtained from Frederick Cancer Center Animal Facilities (National Cancer Institute, Frederick, Md.). The mice were housed in filter-top cages in a laminar flow hood in pathogen-free conditions with 12 hours light/12 hours dark cycles. Animal care was in accordance with the guidelines of NIH Animal Research Advisory Committee.

Human Prostate Cancer Xenograft and Treatment

A human prostate cancer model was established in nude mice by subcutaneous injection of 4×10⁶ DU145 cells in 150 μL of PBS plus 0.2% human serum albumin into the right flank. Palpable tumors developed within 34 days. The mice then received injections of excipient (0.2% human serum albumin in PBS) or chimeric toxin either intraperitoneally (I. P.; 500 μl) or intratumorally (I. T.; 30 μl) using a 27-gauge needle.

Statistical Analysis

Tumor sizes were calculated by multiplying length and width of tumor on a given day. The statistical significance of tumor regression was calculated by Student t test.

B. Results of the Studies Reported in this Example

IL-13R mRNA Expression on Prostate Cancer Cells

Five prostate cancer cell lines, two normal prostate cell lines, and the IL-13Rα2 chain-transfected DU145 cell line (DU145α2) were examined for the expression of IL-13R subunits. The mRNA expression of IL-13R components, IL-13Rα2 and IL-13Rα1 chains was examined by RT-PCR. mRNA for IL-13Rα1 chain was present in all of the cell lines examined. However, no prostate cancer or normal prostate cell lines showed the presence of IL-13Rα2 mRNA except for DU145α2 cells transfected with IL-13Rα2 cDNA. PM-RCC cells that express IL-13Rα2 and IL-13Rα1 mRNA served as a positive control (Murata et al., Biochem Biophys Res Commun., 238:90-94 (1997)).

IL-13 binding to DU145 Cells Increased after Transfection with IL-13Rα2 Chain

The expression and binding affinity of IL-13R on the DU145 cell line was then determined by ¹²⁵I-IL-13 binding assays. DU145 cells do not express IL-13Rα2 chain and therefore show limited binding to ¹²⁵I-IL-13. However, when these cells were transfected with IL13Rα2 chain, consistent with the expression of mRNA for this chain, the binding activity of ¹²⁵I-IL-13 was greatly increased. This binding activity was displaced by an excess of unlabeled IL-13. Because IL-13R and IL-4R share two chains with each other, it was also examined whether IL-4 can also displace the IL-13 binding in DU145 cells transfected with IL-13Rα2 chain (Murata et al., Int J Mol Med., 1:551-557 (1998); Murata et al., Blood, 91:3884-3891 (1998)). IL-4 showed only minimal displacement of ¹²⁵I-IL-13 binding. These findings indicate that IL-13Rα2 chain transfected cells, DU145α2 form type I IL-13 receptors (Obiri et al., J Biol Chem., 270:8797-8804 (1995); Murata et al., Int J Mol Med., 1:551-557 (1998); Murata et al., Biochem Biophys Res Commun., 238:90-94 (1997); Murata et al., Blood, 91:3884-3891 (1998)). To further characterize the IL-13R in IL-13Rα2 chain transfected cells, Scatchard analysis was performed on DU145α2 cells. DU145α2 cells bound IL-13 in a concentration-dependent manner. Scatchard analysis of the binding data showed a single binding site receptor with a Kd value of 1.69±0.4 μM. The number of IL-13Rs was calculated as 15,600±550 IL-13 molecules bound/cell (mean±SD, n=2). Since IL-13 binding sites on vector only transfected DU145 cells were calculated to be 30±5/cell, the increase in IL-13 binding sites in IL-13Rα2 chain transfectants was 520-fold higher compared with control cells.

Prostate Cancer Cells Transfected with IL-13Rα2 Chain Dramatically Increased Sensitivity to IL-13 Toxin

A chimeric protein composed of IL-13 and a truncated form of Pseudomonas exotoxin (IL13-PE38QQR), which was found to be potently cytotoxic to IL-13R-positive solid tumor cells (Debinski et al., J Biol Chem., 270:16775-16780 (1995); Puri et al., Blood, 87:4333-4339 (1996); Husain et al., Clin Cancer Res., 3:151-156 (1997); Husain et al., Blood, 95:3506-3513 (2000)). To determine whether introduction of IL-13Rα2 chain can increase the sensitivity of prostate cancer cell lines to IL-13 toxin, the cytotoxicity of this molecule to normal and prostate cancer cells with or without transfection with IL-13Rα2 chain was evaluated. IL-13 toxin was not cytotoxic to vector only transfected DU145 (DU145mc) cells, however, IL-13Rα2 chain transfected DU145 (DU145α2) cells showed dramatically improved sensitivity to IL-13 toxin. The IC₅₀s of IL-13 toxin to DU145α2 cells improved more than 250-fold compared with vector only transfected DU145mc cells (4.0±0.5 ng/mL vs>1000 ng/mL). Similar to the binding data, the cytotoxic activity of IL13-PE38QQR was neutralized by excess IL-13 but not by IL-4, indicating that cytotoxicity mediated by IL-13 toxin is specific.

Four prostate cancer cell lines and two normal prostate cell lines were also transiently transfected with IL-13Rα2 chain and cytotoxic activity of IL-13 toxin was assessed. Transfection of IL-13Rα2 chain improved sensitivity to IL-13 toxin in all of the cell lines examined. Although the improvement in IC₅₀ was not as dramatic as compared with stably transfected DU145α2 cell line, more than 10-fold to 1000-fold increase in IC₅₀s were observed. Of note, even the normal prostate cell lines (560NPTX and 570NP2TX) can be sensitized to the cytotoxic effect of IL-13 toxin after gene transfer of IL-13Rα2 chain

Antitumor activity of IL-13 toxin to prostate cancer dramatically enhances after gene transfer of IL-13Rα2 chain

It was found that transfection of IL-13Rα2 chain improved the sensitivity of prostate cancer cell lines to the cytotoxic effect of IL-13 toxin. To explore whether these findings could be applied to an in vivo tumor model, groups of nude mice were injected subcutaneously with growing DU145mc cells or with DU145 cells transfected with IL-13Rα2 chain (DU145α2). After the tumors had established, the animals were injected intraperitoneally with IL13-PE38QQR or with excipient only (as a control).

The animals injected with DU145mc tumor cells had rapidly growing tumors. Animals were then treated with IL-13 toxin (50 μg/kg) twice daily for 5 days (total 10 injections) from day 6 to day 10 showed no antitumor efficacy as measured by tumor size over a 60-day period. On day 60, mice in both groups (n=5) were sacrificed due to large tumor burden.

On the other hand, when animals with DU145 tumors transfected with IL-13Rα2 chain (DU145α2) were treated with IL-13 toxin (50 μg/kg) or with excipient only, on the same schedule (twice daily for 5 days) as for the first group. Four out of 5 mice showed complete regression by day 11. By day 22, palpable tumors recurred in all of these mice, however, the size of tumors remained significantly smaller compared to the excipient injected mice (P<0.0002). The average size of the tumors remained smaller than the size before injection until day 43 (27 mm²). On day 60, the size in the treated group was 68% less compared to mice in the excipient-only injected group.

Complete Regression of IL-13Rα2 Chain Gene Transferred Prostate Tumors by Intratumoral Administration of IL-13 Toxin

It has previously been observed that availability of drug at the tumor site is of great importance in treating tumors in mice. To achieve a higher accumulation of drug, IL-13 toxin was directly injected into the tumor bed (Husain et al., Clin Cancer Res., 3:151-156 (1997); Husain et al., Blood, 95:35063513 (2000)). As expected, there were more complete responders with intratumoral injection of IL13-PE38QQR into IL-13Rα2 gene transferred prostate cancer, DU145α2 tumors compared to intraperitoneally injected tumor groups. After three injections of IL-13 toxin (250 μg/kg per day on alternate days beginning on day 3), two of four tumors disappeared completely by day 7. By the day of the last injection (day 10), 100% of the tumors were completely regressed. Although by day 24 tumors recurred in two mice, two other mice remained tumor free until day 90 (data not shown).

Partial Responder Prostate Tumors Retain Sensitivity to IL-13 Toxin

To determine whether DU145α2 tumors that recurred in L13-PE38QQR treated animals maintained sensitivity to IL-13 toxin, tumors were resected from both intraperitoneally injected vehicle only and IL-13 toxin treated (50 μg/kg; twice daily for 5 days from day 6 and day 10) mice from day 10 and day 60 after the implantation of tumor. Tumors were minced in pieces and digested with cocktail of 10 μg/mL collagenase, 1 mg/mL hyaluronidase, and 0.5 mg/mL DNAse (Sigma Chemical Co., St. Louis, Mo.). Tumor cells were cultured in EMEM medium containing 10% fetal bovine serum. After three passages, cell debris and contaminating blood cells were removed, and cells were assessed for sensitivity to IL-13 toxin. All of the cell lines maintained sensitivity to IL13 toxin. The IC₅₀ in all four tumor cells remained close to the IC₅₀ (4±0.5 ng/mL) of the transfected cell line injected to establish tumors.

C. Discussion of the Studies Reported in this Example

The results reported here demonstrate that gene transfer of IL-13Rα2 chain gene into low level or no IL-13Rα2 chain expressing prostate tumor cells dramatically sensitized the cells towards IL-13R-targeted cytotoxin in vitro and in vivo.

It is noteworthy that after gene transfer of IL-13Rα2 chain into prostate cancer cell lines as well as normal prostate cell lines an enhancement to the cytotoxic activity to IL-13 toxin was observed. This observation suggests that the activity of IL-13 toxin was specific to cells that express IL-13Rα2 chain. When cells such as DU145 were stably transfected with IL-13Rα2 chain, (to form, for example, DU145α2 cells), the expression of IL-13R binding sites and cytotoxic activity of IL-13 toxin was dramatically increased. These in vitro results also translated into an in vivo DU145α2 xenograft model. A dramatic increase in the antitumor activity of IL-13 toxin was achieved. A 68% reduction in tumor size was observed after intraperitoneal treatment with IL-13 toxin. Because IL-13 toxin, IL-13-PE38QQR, is found to be specific to IL-13R expressing cancer cells and not to human T and B cells, monocytes, normal endothelial cells, and resting or growth factor-activated bone marrow cells that do not express IL-13Rα2 chain, (Puri et al., Blood, 87:4333-4339 (1996); Murata et al., Biochem Biophys Res Commun., 238:90-94 (1997)) these findings offer wide possibilities for the utilization of IL-13 toxin in prostate and other cancers that are normally insensitive to IL-13 targeted immunoconjugates.

It has been found that various tumor cell lines including renal cell carcinoma, glioblastoma, and AIDS-associated Kaposi's sarcoma express high levels of receptors for UL-13 (Debinski et al., J Biol Chem., 270:16775-16780 (1995); Puri et al., Blood, 87:43334339 (1996); Husain et al., Clin Cancer Res., 3:151-156 (1997); Husain et al., Blood, 95:3506-35.13 (2000); Joshi et al., Cancer Res. 60:1168-1172 (2000)). These receptors were found to be a novel target for IL-13R-targeted cytotoxin therapy. Although prostate cancer cells express functional IL-13R, their potential as a target for IL-13R targeted cytotoxin therapy was not initially promising. The gene transfer of one subunit of cytokine receptor chain into prostate cancer cells dramatically increased their sensitivity to IL-13 toxin.

### Example 4

This Example shows the heterogenity of expression the IL-13R in squamous cell carcinoma of the head and neck (“SCCHN”) and whether differences in expression level account for the differences in sensitivity of SCCHN cells to IL-13-targeted chimeric toxins.

A. Materials and Methods Used in the Studies Reported in this Example

Cell culture: SSCHN cell lines KB, A253, RPMI 2650, and Hep-2 were purchased from the American Type Culture Collection (Manassas, Va.). The WSU-HN12 (12) cell line was a kind gift from Dr. Andrew Yeudall (National Dental and Craniofacial Research Institute, NIH, Bethesda, Md. (Cardinali, M. et al., Int J. Cancer, 61:98-103 (1995)). Twelve head and neck cell lines were established in the Department of Otolaryngology, Yokohama City University School of Medicine Research Institute, Kanagawa Cancer Center, Yokohama, Japan (Kawakami, K. et al., Anticancer Res., 19:3927-32 (1999)). These cell lines were maintained either in Eagle's Modified Essential Medium (KB, A253, Hep-2, RPMI 2650 and HN12) or RPMI 1640 (twelve cell lines from Yokohama University, Japan) containing 10% fetal bovine serum (Bio-Whittakar Inc., Walkerville, Md.), 1 mM HEPES, 1 mM nonessential amino acids, 100 μg/ml penicillin and 100 μg/ml streptomycin Bio-Whittakar Inc., Walkerville, Md.).

RNA extraction: SSCHN cells in the logarithmic phase were detached with Trypsin-EDTA, washed with 1× PBS and RNA was extracted using RNaeasy RNA extraction kit (Qiagen, Valencia, Calif.) according to manufacturer's instructions. Briefly, 10×10⁶ cells were pelleted and lysed in guanidium-thiocyanate lysis buffer. The total cell lysate was mixed with an equal volume of 70% ethanol and loaded on silica spin columns. After a brief centrifigation for 20 sec, the columns were washed and RNA eluted with RNase-free water. RNA was quantitated.

RT-PCR: Seventeen RNA samples from SCCHN cells were subjected to RT-PCR analysis. β-actin mRNA amplification from these samples served as an internal control. RT-PCR conditions for each chain and the primers used in the amplification protocols have been published previously Murata, T. et al., Biochem Biophys Res Commun., 238:90-4 (1997). Five hundred nanograms of total RNA from these cell lines were reverse-transcribed using a RNA-PCR kit according to the manufacturer's instructions (Perkin-Elmer Corp., Norwalk, Conn.). Ten microliters of the reverse-transcribed products were amplified for 30 cycles using the GeneAmp® PCR system 9700 ( Applied Biosystem-Perkin Elmer, Norwalk, Conn.)). The amplified products were electrophoresed on 2% agarose gel, stained with ethidium bromide, visualized in a transilluminator, and photographed.

Immunofluorescence Analysis: Twenty thousand cells were cultured in a chambered glass slide (Lab Tek-Nagle Nunc International, Naperville, Ill.) for 48 hours. The cells were washed twice with 1× PBS and fixed with cold methanol:acetone (1:1, v/v) and incubated at −20° C. for 2 h. The cells were then washed and rehydrated with PBS and subjected to immunofluorescence analysis. The optimal conditions for immunofluorescence analysis were previously described (Joshi, B. H. et al., Cancer Res., 60:1168-72 (2000)). Briefly, the rehydrated cells were incubated with 1% BSA and 5% goat or horse serum in PBS to block nonspecific binding of antibody. The slides were washed with PBS twice and incubated for two hours with either the specified primary antibody (1:1500) or mouse IgG1 or rabbit IgG as isotype control. Slides were then washed three times and incubated for 1 h with a secondary antibody that had either tetramethylrhodamnine isothiocyanate or FITC tag after diluting in PBS containing 0.1% BSAper manufacturer's instructions. The slides were washed with PBS three times, air dried and layered with Vectashield antifluorescence fading mounting medium (Vector Laboratories, Burlingame, Calif.) and a coverslip. The slides were viewed in a Nikon fluorescence microscope using appropriate filters.

IL-13 receptor binding studies: Recombinant human IL-13 was labeled with ¹²⁵I (Amersham Research Products,) by using IODO-GENO reagent (Pierce, Rockford, Ill.) according to the manufacturer's instructions. The specific activity of the radiolabeled cytokine was estimated to range between 40-120 μCi/μg of protein. Binding experiments were performed as described elsewhere (Obiri, N. I. et al., J Biol Chem., 270:8797-804 (1995)). Typically, 1×10⁶ cells were incubated at 4° C. for 4 h with ¹²⁵IL-13 (100-500 pM).in the absence or presence of 200 fold unlabeled IL-13. Duplicate samples of the cells associated with ¹²⁵I-IL-13 were separated from free ¹²¹I-IL-13 by centrifiugation through cushion of phthalate oils. The cell pellets were counted in a Gamma-Counter (Wallac, Gaithersburg, Md.). The binding sites were calculated using specific activity of IL-13.

Construction of IL-13PE chimeric gene: The IL-13 and Psuedomonas exotoxin 38 (PE38) and IL-13 Pseudomonas exotoxin 38QQR (PE38QQR) chimeric genes were constructed in the laboratory. Briefly, the hIL-13 gene was cloned in its matured form from stimulated human PBMCs. Total RNA was extracted from PBMCs and reverse-transcribed to cDNA with MuMLV reverse-transcriptase. PCR based amplification of cDNA was performed to produce the IL-13 gene with Nde I and Hind III sites at 5′ and 3′ of the ORF of gene by using sequence specific primers. A 336-base pair long DNA fragment was purified from the PCR product and digested with the appropriate restriction enzymes. The digested DNA fragment was sub cloned into the vector obtained from previously digested plasmid YR39 or pRKL438QQR (kindly provided by Dr. Ira Pastan, National Cancer Institute, Bethesda, Md.) with the same restriction endonuclease enzyme pair to yield IL-13-PE38 and IL-13-PE38QQR. The junctions of the chimeric genes as well as IL-13 genes were sequenced to confirm correct DNA sequence.

Expression and Purification of the chimeric proteins: Expression and purification of IL-13-PE38 and IL-13-PE38QQR was carried out using E.coli BL21(λDE3)pLys for transformation. The bacterial culture was induced with 1 mM isopropyl-β-D-thiogalactopyranoside (IPTG) and placed in a bacterial shaker for six hours. The chimeric proteins were produced in inclusion bodies. After washing, the inclusion bodies were denatured with guanidinium-hydrochloride containing Tris-HCl buffer pH 8.0 overnight Soluble inclusion bodies were refolded by diluting 1:150 with Tris-HCl buffer containing arginine and oxidized glutathione. The renatured preparation was dialysed against 10 mM Tris-Cl pH 7.4 buffer containing 60 mM urea. The chimeric protein was purified by Fast Protein Liquid Chromatography using Q Sepahrose, mono Q and sephacryl S-100 gel exclusion columns (Amersham Pharmacia,Piscataway, N.J.). The purified protein was electrophoresed on 10% SDS-PAGE and stained with Coomassie Blue. The gel was destained with destaining solution that contained 7% acetic acid and 5% methanol (v/v).

Protein synthesis Inhibition Assay: The cytotoxicity of chimeric toxins IL-13-PE38 and IL-13-PE38QQR was determined as described previously (Puri, R. K. et al., Cancer Res., 51:3011-7 (1991)). Briefly, 1×10⁴ cells were plated in leucine free medium Biofluids, Rockville, Md.) for 6 h to allow adherence to flat-bottom microtiter plates. Various concentrations of either cytotoxin were added to the cells and incubated for 20 h at 37° C. For blocking experiments, cells were pre incubated with IL-13 or IL-4 (2kg/ml) for 45 min before addition of IL-13 toxin. 1 μCi of [³H]leucine (NEN Research Products, Boston, Mass.) was then added to each well and the cells were incubated for an additional 4 h. Cells were harvested and labeled leucine incorporation into cells was measured by a P plate counter (Wallac Gaithersburg, Md.).

Transient transfection of IL-13Rα2 DNA: YCUMS861 and KB cell lines were plated onto 100-mm petri dish and grown until the plate was 60% confluent. Human IL-13Rα2 cDNA (Caput, D. et al., J Biol Chem., 271:16921-6 (1996)) was cloned into a pME18S expression vector for transient transfection experiments. Plasmid DNA (12 μg/100-mm petri dish) of each cell line was transfected with Gene Porter™ transfection reagent (Gene Therapy Systems, San Diego, Calif.) according to the manufacturer's instructions. In brief, 3×10⁶ cells were cultured with DNA-GenePorter™ mixture for 5 h in DMEM. DMEM containing 20% FBS was added and the culture was maintained for an additional 48 h with one change of medium.

B. Results of the Studies Reported in this Example

Subunit structure and characterization of IL-13R: The molecular configuration of IL-13R on 17 SCCHN cell lines was examined by RT-PCR analysis for different receptor chains. As shown in Table 4, three of 17 SCCHN cell lines strongly expressed mRNA for IL-13Rα2 chain while 5 other cell lines (YCUL891, KCCTC871, KCCL871, KCCTCM901, and RPAI 2650) expressed very low levels. The RT-PCR results for H-13Rα2 chain was correlated with the site of tumor origin as shown in Table 5. Although the sample size was small, the results suggest that 2 of 3 (67%) of larynx, 2 of 4 (50%) tongue and 1 of 3 (33%) pharynx, and one of one (100%) lymph node originated cell lines expressed low to high levels of IL-13Rα2 chain mRNA. On the other hand, IL4Rα and IL-13Rα1 chain mRNAs were uniformly present in all 17 cell lines, except YCUL891 and YCUM862 that appeared to show stronger band. None of the SCCHN cell lines showed RT-PCR positivity for the presence of γ_{c} mRNA that is abundantly present in H9 T lymphoma cells that served as a positive control.

Immunofluorescence analysis for receptor subunit protein on SCCHN cell lines: Next, the expression of different receptor proteins was examined by indirect immunofluorescence analysis in high and low IL-13Rα2 chain expressing SCCHN cell lines. Fluorescence positivity was shown for IL-13Rα2 protein the high expresser cell lines,YCUM911, HN12 and KCCT873 cells. On the other hand, IL-13Rα2 -negative cell line did not show any fluorescence positivity. These results correlated with PCR positivity for their corresponding mRNAs in these cells. Immunofluorescence expression of the IL-13Rα1 and IL-4Rα chains in SCCHN cell lines demonstrated that these two chains are expressed intracytosolically as well as on the cell surface. However, similar to RT-PCR results, none of these cell lines expressed γ_{c} protein.

Expression of IL-13R on SCCHN cells: On the basis of RT-PCR and immunofluorescence staining results, it was thought that radiolabeled IL-13 would specifically bind to SCCHN cell lines. Therefore, binding studies were performed using ¹²⁵I-IL-13 in three IL-13Rα2 expressing cell lines. As shown in Table 6, these three cell lines expressed high number of IL-13 binding sites on their cell surface. The number of IL-13 binding sites ranged between 5800 and 8600 per cell in these cell lines.

Generation of IL-13-Pseudomonas exotoxin fusion genes and proteins: In order to generate a chimeric construct of IL-13 with a mutated form of Psuedomonas exotoxin, the ORF of the L-13 gene was ligated with domains II and III of Pseudomonas exotoxin (IL-13-PE38QQR or IL-13-PE38) and placed in a pET vector. These chimeric genes were used to transform E. coli. Upon IPTG induction, chimeric fusion proteins were induced and purified to high purity by FPLC. Both proteins appeared to be induced equally well with IPTG and purified to single band entities demonstrating high purity of the protein products. The chimeric proteins migrated approximately at 52 kDa as expected.

Cytotoxic activity of IL-13 toxins in SCCHN cell lines: The cytotoxic activity of IL-13-PE38QQR on SCCHN cell lines was first tested by protein synthesis inhibition assays. IL-13-PE38QQR was highly cytotoxic to three IL-13Rα2-positive SCCHN cell lines. The IC₅₀ (concentration of IL-13 toxin causing 50% inhibition of protein synthesis) ranged between 4-9 ng/ml. PM-RCC cell line that has been shown to express high numbers of IL-13R, was extremely sensitive to IL-13-PE38QQR (IC₅₀, 0.1 ng/ml) (Puri, R. K. et al., Blood, 87:4333-9 (1996)). The 14 cell lines that lacked or expressed very low levels of IL-13Rα2 chain by RT-PCR were considerably less sensitive to IL-13-PE38QQP, The IC₅₀in these cell lines ranged between 100-1000 ng/ml (Table 7). The specificity of IL-13 toxin mediated cytotoxicity was confirmed by neutralization assays in the presence of excess of IL-13 or IL-4. In all three cell lines, IL-13 was able to neutralize cytotoxic activity, while IL-4 did not, indicating specificity.

The cytotoxic activity ofIL-13-PE38QQR was next compared with L-13-PE38, which was produced by an identical technique. As shown in Table 76, IL-13-PE38 was equally cytotoxic to IL-13Rα2-positive cell lines when compared with IL-13-PE38QQR (IC₅₀<10 ng/ml) while it was less cytotoxic or not cytotoxic to other 14 SCCHN cell lines (IC₅₀, 100-1000 ng/ml).

C: Increased Sensitivity of SCCHN Cell Lines upon Gene Transfer of IL-13Rα2 Chain:

As only a few SCCHN cell lines were highly sensitive and the majority of the cell lines were modestly sensitive or not sensitive at all, we examined whether not sensitive or modestly sensitive cell lines could be sensitized to high cytotoxic effect of IL-13 toxin. IL-13Rα2 chain cDNA was transiently introduced into YCUMS861 and KB cell lines to determine if this could increase their sensitivity to IL-13 toxin. Transfection of IL-13Rα2 chain in YCUMS861 and KB cell lines improved their sensitivity to IL-13-PE38QQR. The IC₅₀ in YCUM861 SCCHN cell line decreased by 12 fold from 1000 ng/ml to 80 ng/ml and from 125 ng/ml to 10 ng/ml in KB cell line as compared to mock-transfected control cells.

D. Discussion of the Results of the Studies Reported in this Example:

The results of the studies reported herein indicated that 20% of human SCCHN cell lines express high density IL-13R at mRNA and protein levels. The high level of receptor expression correlated with the expression of the primary IL-13 binding protein, IL-13Rα2 chain. Cell lines that were weakly positive for this chain express few IL-13R. On the other hand, all 17 SCCHN cell lines expressed IL-13Rα1 and IL4Rα chains. Since IL-13Rα1 and IL-4Rα chains are required for IL-4 or IL-13 induced signal transduction (Murata, T. et al., Int J. Cancer., 70:230-40 (1997); Murata, T. et al., Int J Mol Med., 1:551-7 (1998); Murata, T. et al., Cellullar Immunology, 175:3340 (1997); Murata, T. et al., Blood, 91:3884-91 (1998); Murata, T. et al., J Immunol., 156:2972-8 (1996); Murata, T. et al., Int Immunol., 10:1103-10 (1998); Orchansky, P. L. et al., J Biol Chem., 274:20818-25 (1999); Zurawski, S. M. et al., J Biol Chem., 270:13869-78 (1995)), the results suggest that SCCHN cell lines express functional IL-13R. These results also indicate that SCCHN cell lines express two types of IL-13R. Twenty percent of cell lines expressed type I IL-13R while 50% expressed predominantly type II IL-13R Another 30% cell lines possibly also expressed type I IL-13R. As none of the SCCHN cell lines expressed γ_{c} chain, no type III IL-13R were observed. The results further indicate the phenotypic heterogeneity of SCCHN as defined by IL-13R expression. Upon further analysis of the data, it was found that 50% of SCCHN tumors derived from tongue, 67% derived from larynx, 33% derived from pharynx and one tumor derived from lymph node expressed ILI13Rα2 chain, suggesting that the origin of tumors may determine IL-13R configuration.

It is of interest to note that the 20% of SCCHN cell lines that expressed mRNA and protein for IL-13Rα2 chain were highly sensitive to the cytotoxic effect of IL-13-PE38QQR. The other 80% cell lines showed low or no sensitivity. The difference in the IC₅₀ between IL-13Rα2-positive cell lines and negative cell lines ranged between 11-fold and 110 fold. IL-13-PE38QQR has been shown to be highly cytotoxic to a variety of solid human tumor cell lines e.g. renal cell carcinoma (Puri, R. K. et al., Blood, 87:4333-9 (1996)), AIDS associated Kaposi's Sarcoma (Husain, S. R. et al., Clin Cancer Res., 3:151-6 (1997)) and malignant glioma (Debinski, W. et al., Clin Cancer Res., 1:1253-8 (1995)). The current results extend the list of IL-13-PE38QQR responsive tumors. Since IL-13Rα2-positive tumor cell lines were found to be responsive to IL-13PE38QQR, the results suggest that IL-13Rα2 is predominantly responsible for IL-13 toxin induced cytotoxicity in SCCHN tumors. These results further confirm that IL-13Rα2 chain alone is sufficient to internalize the IL-13-IL-13R complex. In addition, this chain alone is sufficient to sensitize cancer cells to the cytotoxic activity of IL-13 toxin. This conclusion is confirmed by the results of transient gene transfer of IL-13Rα2 chain in two different IL-13Rα2-negative SCCHN cell lines. These transfectants acquired sensitivity to IL-13 toxin in vitro.

In previous studies, IL-13-PE38QQR has been utilized in vitro and in vivo for targeting IL-13R positive tumors (Debinski, W. et al., Clin Cancer Res., 1:1253-8 (1995); Husain, S. R. et al., Clin Cancer Res., 3:151-6 (1997); Debinski, W. et al., J Biol Chem., 270:16775-80 (1995); Puri, R. K et al., Blood, 87:4333-9 (1996). In this fusion molecule, the C-terminus of the IL-13 molecule was fused to the N-terminus of domain II of the PE molecule. In addition, lysines at position 509 and 606 and arginine at position 613 in PE molecules were substituted by glutamine and lysine (PE38QQR). Since the role of these mutations in the IL-1 3-PE molecule has not been delineated, here these mutations were deleted and produced PE38. IL-13-PE38 was expressed in E. coli in an identical manner to IL,13-PE38QQR. Upon in vitro testing, IL-13-PE38 produced results identical to use of IL-13-PE38QQR, indicating that the 3 amino acid mutation at C-terminus of PE has no effect on IL-13-PE38 mediated cytotoxicity in the tumor cells tested.

In short, the incidence and occurrence of IL-13R in SCCHN cell lines vary with the site of origin of tumor. Varying number of tumors from tongue, larynx and pharynx have been found to express mRNA and protein for IL-13Rα2 chain. As IL-13-PE38 and IL-13-PE38QQR are highly cytotoxic to IL-13Rα2-positive SCCHN cell lines, EL-13R can serve as a target for delivery of cytotoxins to the certain type of SCCHN tumors. For SCCHN tumors that lack IL-13Rα2 chain, gene transfer of this chain may sensitize them to the cytotoxic effect of IL-13PE. Various approaches of gene transfer have been tested in vivo (Agha-Mohammadi, S. and Lotez, M. T., J Clin Invest., 2000:1173-1176 (2000); Pick, J. E., Nat Med., 6:624-626 (2000); Marchisone, C. et al., J Exp Clin Cancer Res., 19:261-70 (2000)). Among them, plasmid mediated or virus mediated gene transfer may be most desirable.

TABLE 4
mRNA expression for different receptor subunits in SCCHN cell lines.

| | | Receptor subunit^{a} | | | |
|---|---|---|---|---|---|
| Cell type | Origin | α2 | α1 | IL-4Rα | γ_{c} |
| 1. YCUMS861 | Maxillary sinus | − | ++ | ++ | − |
| 2. KCCT871 | Tongue | ± | ++ | ++ | − |
| 3. KCCT891 | Hypopharynx | − | ++ | ++ | − |
| 4. KCCL871 | Larynx | ± | ++ | ++ | − |
| 5. KCCOR891 | Oral floor | − | ++ | ++ | − |
| 6. YCUL891 | Larynx | ± | ++ | ++ | − |
| 7. YCUM862 | Oropharynx | − | ++ | ++ | − |
| 8. YCUM911 | Oropharynx | +++ | ++ | ++ | − |
| 9. YCUT891 | Tongue | − | ++ | ++ | − |
| 10. YCUT892 | Tongue | − | ++ | ++ | − |
| 11. KCCTCM901 | Metastasis to the | ± | ++ | ++ | − |
| | chest fluid | | | | |
| 12. KCCT873 | Tongue | ++ | ++ | ++ | − |
| 13. A253 | Submandibular gland | − | ++ | ++ | − |
| 14. HN12 | Lymph node | +++ | ++ | ++ | − |
| 15. KB | Mouth | − | ++ | ++ | − |
| 16. Hep-2 | Larynx | − | ++ | ++ | − |
| 17. RPMI2650 | Nasal Septum | ± | ++ | ++ | − |

^{a} Positivity of the RT-PCR product was ascertained by fluorescence intensity after staining with ethidium bromide.
−, negative;
±, weakly positive;
++, strongly positive;
+++, more strongly positive.

TABLE 5
Incidence of IL-13Rα2 positivity in SCCHN cells

| | |
|---|---|
| No. of cell lines with | |
| IL-13Rα2 positivity | Positivity |

| Origin | Total | − | ± | ++ | +++ | (%) |
|---|---|---|---|---|---|---|
| Tongue | 4 | 0 | 1 | 1 | 0 | 50.0 |
| Larynx | 3 | 1 | 2 | 0 | 0 | 66.6 |
| Pharynx^{a} | 3 | 2 | 0 | 0 | 1 | 33.3 |
| Maxillary Sinus | 1 | 1 | 0 | 0 | 0 | 0 |
| Oral Floor | 1 | 1 | 0 | 0 | 0 | 0 |
| Meta.^{b} | 1 | 1 | 0 | 0 | 0 | 0 |
| Submand^{c}. | 1 | 1 | 0 | 0 | 0 | 0 |
| Lymph Node | 1 | 0 | 0 | 0 | 1 | 100.0 |
| Mouth | 1 | 1 | 0 | 0 | 0 | 0 |
| Nasal Septum | 1 | 1 | 0 | 0 | 0 | 0 |

Data from Table 4 was regrouped with the origin of SCCHN.
^{a}tumor originated at Oro or hypo pharynx
^{b}tumor originated from metastatis to Chest Fluid
^{c}tumor originated at submandibular gland

TABLE 6
IL-13 receptor expression on SCCHN cells

| | | IL-13-PE38QQR |
|---|---|---|
| cell type | IL-13 binding sites/cell^{a} | IC₅₀(ng/ml)^{b} |
| 1. HN12 | 5800 ± 203 | 7.5 ± 1.2 |
| 2. YCUM911 | 8600 ± 112 | 4.5 + 0.32 |
| 3. KCCTC873 | 6185 ± 282 | 8.6 ± 1.8 |

^{a}number of binding sites for each cell type were calculated by radioreceptor binding as described in Materials and Methods.
The data are shown as the mean no. of molecules/cell ± S.E. of
three experiments performed in quadruplicate.
^{b}IC50, the concentration of IL-13 toxin at which 50% inhibition
of protein synthesis is observed compared with untreated cells.
The data are shown as the mean ± S.D. of two experiments performed
in quadruplicate.

TABLE 7
Cytotoxic activity of IL-13-PE38 and
IL-13-PE38QQR in SCCHN cell lines.

IC₅₀(ng/ml)^{a}

| Cell type | | IL-13PE38 | IL-13PE38QQR |
|---|---|---|---|
| 1. | YCUMS861 | ND | ND |
| 2. | KCCT871 | 275.0 | 300.0 |
| 3. | KCCT891 | >1000.0 | >1000.0 |
| 4. | KCCL871 | 185.0 | 200.0 |
| 5. | KCCOR891 | ND | ND |
| 6. | YCU891 | 500.0 | 500.0 |
| 7. | YCUM862 | >1000.0 | >1000.0 |
| 8. | YCUM911 | 4.0 | 4.5 |
| 9. | YCUT891 | >1000.0 | >1000.0 |
| 10. | YCUT892 | >1000.0 | >1000.0 |
| 11. | KCCTCM901 | 110.0 | 100.0 |
| 12. | KCCTC873 | 8.0 | 8.6 |
| 13. | A253 | 155.0 | 150.0 |
| 14. | HN12 | 7.5 | 7.5 |
| 15. | KB | 100.0 | 200.0 |
| 16. | Hep-2 | 200.0 | 200.0 |
| 17. | RPMI2650 | >1000.0 | >1000.0 |

^{a}IC50, concentration of IL-13 toxin at which 50% inhibition of protein synthesis is achieved as compared with untreated cells.

### Example 5

Athymic nude mice 4 weeks old (about 20 g in body weight) were obtained from Frederick Cancer Center Animal Facilities (National Cancer Institute, Frederick, Md.). The mice were housed in filter-top cages in a laminar flow hood in pathogen-free conditions with 12 hours light/12 hours dark cycles. Animal care was in accordance with the guidelines of NIH Animal Research Advisory Committee.

Cells of a commonly used pancreatic cancer cell line, PANC-1, were transfected with the IL-13Rα2 chain, in a manner similar to that described in the previous Examples. Other cells of the same cell line were mock-transfected. The nude mice were divided into two groups, experimental and control, and were inoculated on the flanks with equal numbers of transfected PANC-1 cells (the experimental group of mice) or with the mock transfected cells (the control group). The mock transfected cells grew robustly into large tumors. In contrast, the PANC-1 cells transfected with the IL-13Rα2 chain did not grow. It was concluded that the presence of the IL-13Rα2 chain alone in cells of this cancer inhibited cell growth even in the absence of contacting with an IL-13R-targeted immunoconjugate.

### Example 6

Athymic nude mice 4 weeks old (about 20 g in body weight) were obtained from Frederick Cancer Center Animal Facilities (National Cancer Institute, Frederick, Md.). The mice were housed in filter-top cages in a laminar flow hood in pathogen-free conditions with 12 hours light/12 hours dark cycles. Animal care was in accordance with the guidelines of NIH Animal Research Advisory Committee.

Cells of a widely used breast cancer cell line, MDA-MB-231, were transfected with the IL-13Rα2 chain, in a manner similar to that described in the previous Examples. Other cells of the same cell line were mock-transfected. The nude mice were divided into two groups, experimental and control, and were inoculated on the flanks with equal numbers of transfected MDA-MB-231 cells (the experimental group of mice) or with the mock transfected cells (the control group). The mock transfected cells grew robustly into large tumors. In contrast, the MDA-MB-231 cells transfected with the IL-13Rα2 chain did not grow. It was concluded that the presence of the IL-13Rα2 chain alone in cells of this cancer inhibited cell growth even in the absence of contacting with an IL-13R-targeted immunoconjugate.

### Example 7

This Example reports the results of studies regarding the transfection of tumor cells in vivo, and subsequent systemic or intratumoral administration of an exemplary IL-i 3R-targeted immunotoxin.

Head and neck cancer cell line A253 or prostate tumor cells DU145 were implanted in the flanks of nude mice on day 0 and permitted to establish tumors. When palpable tumors developed (days 34), 25 μg of a cDNA plasmid vector encoding the IL-13Rα2 chain, in 20 mM of N-(1-[2, 3-dioleoyloxy]propyl)-N, N, N-trimethylammonium chloride (DOTAP):Cholesterol (1:1 molar ratio) liposome (Sigma-Aldrich, Inc., St. Louis, Mo.) was injected intratumorally. The formulation was injected on three consecutive days (days 4,5, and 6). Immunotoxin IL13-PE38QQR in an excipient of 0.2% human serum albumin in phosphate buffer saline, or the excipient only, as a control, was then administered either intraperitoneally (“IP,” 500 μl mouse, administered 2 times per day for 5 days, days 5-9) or intratumorally (“IT,” 30 μl/tumor, administered once a day for five days, on days 5-9).

Transfection of cells with IL-13Rα2 chain was confirmed by RT-PCR. It proved difficult to quantitate the percentage of cells that were transfected. Preliminary studies using green fluorescent protein (“GFP”) as a marker indicated that intratumoral transfection by the route used in these studies resulted in transfection of >50% of the cells in the tumor. Based on the preliminary studies using GFP, it is believed that over 50% of the cells in the tumors studied were transfected with IL-13Rα2 chain, but that not all the cells were so transfected.

The tumors that were transfected and exposed to the immunotoxin by IP administration showed remarkable tumor regression. The tumors that were transfected and exposed to the immunotoxin by IT administration showed complete tumor regression.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A use of a vector encoding a polypeptide with at least 70% identity to an amino acid sequence of a IL-13 receptor α2 chain (SEQ ID NO:1) to manufacture a medicament for sensitizing a cancer cell to an immunoconjugate that binds to an IL-13 receptor, provided that said encoded polypeptide can bind IL-13.

2. A use of claim 1, wherein said encoded polypeptide has at least 80% identity to an IL-13 receptor α2 chain (SEQ ID NO:1).

3. A use of claim 1, wherein said encoded polypeptide has at least 90% identity to an IL-13 receptor α2 chain (SEQ ID NO:1).

4. A use of claim 1, wherein said encoded polypeptide has the sequence of IL-13 receptor α2 chain (SEQ ID NO:1).

5. The use of claim 1, wherein said cancer cell is a cell from a cancer selected from the group consisting of: a brain cancer, a head and neck cancer, a breast cancer, a liver cancer, a lung cancer, a mesothelioma, a pancreatic cancer, a colon cancer, a gastric cancer, an ovarian cancer, a renal cancer, a bladder cancer, a prostate cancer, a testicular cancer, a skin cancer, a cervical cancer, a uterine cancer, and a sarcoma.

6. A use of claim 5, wherein said head and neck cancer is a squamous cell carcinoma.

7. A use of a vector encoding a polypeptide with at least 70% identity to an amino acid sequence of a IL-13 receptor α2 chain (SEQ ID NO:1) for the manufacture of a medicament for inhibiting the growth of a cancer cell, provided that said encoded polypeptide can bind IL-13.

8. A use of claim 7, wherein said encoded polypeptide has at least 80% sequence identity to an IL-13 receptor α2 chain (SEQ ID NO:1).

9. A use of claim 7, wherein said encoded polypeptide has at least 90% sequence identity to an IL-13 receptor α2 chain (SEQ ID NO:1).

10. A use of claim 7, wherein said encoded polypeptide has the sequence of IL-13 receptor α2 chain (SEQ ID NO:1).

11. The use of claim 7, wherein said cancer cell is a cell from a cancer selected from the group consisting of a breast cancer and a pancreatic cancer.

12. A composition comprising a nucleic acid encoding a polypeptide with at least 70% identity to an IL-13 receptor α2 chain (SEQ ID NO:1) operably linked to a promoter, and a pharmaceutically acceptable carrier, provided that said encoded polypeptide can bind IL-13.

13. A composition of claim 12, wherein said polypeptide has at least 80% identity to an IL-13 receptor α2 chain (SEQ ID NO:1).

14. A composition of claim 12, wherein said polypeptide has at least 90% sequence identity to an IL-13 receptor α2 chain (SEQ ID NO:1).

15. A composition of claim 12, wherein said polypeptide has the sequence of an IL-13 receptor α2 chain (SEQ ID NO:1).

16. A method for inhibiting the growth of a cancer tumor, said method comprising transfecting at least some cells of said tumor with a nucleic acid sequence encoding a polypeptide with at least 70% identity to an IL-13Rα2 chain (SEQ ID NO:1), provided said encoded polypeptide can bind IL-13.

17. A method of claim 16, wherein said encoded polypeptide has at least 80% identity to an IL-13Rα2 chain (SEQ ID NO:1).

18. A method of claim 16, wherein said encoded polypeptide has at least 90% identity to an IL-13Rα2 chain (SEQ ID NO:1).

19. A method of claim 16, wherein said encoded polypeptide has the sequence of an IL-13Rα2 chain (SEQ ID NO:1).

20. A method of claim 16, wherein the cancer tumor is selected from the group consisting of a pancreatic cancer and a breast cancer.

21. A method for sensitizing a cancer cell to an effector molecule, the method comprising transfecting said cell with a nucleic acid sequence encoding a polypeptide with at least 70% identity to an IL-13Rα2 chain (SEQ ID NO:1), provided said encoded polypeptide can bind IL-13.

22. A method of claim 21, wherein said encoded protein has at least 85% identity to an IL-13Rα2 chain (SEQ ID NO:1), provided said encoded polypeptide can bind IL-13.

23. A method of claim 21, wherein said encoded polypeptide has the sequence of an IL-13Rα2 chain (SEQ ID NO:1).

24. A method of claim 21, further wherein said cell is contacted with an immunoconjugate comprising a targeting moiety and an effector moiety, wherein said targeting moiety is a ligand for the IL-13Rα2 chain (SEQ ID NO:1).

25. A method of claim 24, wherein said ligand is selected from the group consisting of IL-13, a mutated IL-13, which mutated IL-13 retains the ability to bind to an IL-13Rα2 chain (SEQ ID NO:1), a circularly permuted IL-13 (“cpIL-13”), and an antibody that specifically binds to an IL-13Rα2 chain (SEQ ID NO:1).

26. A method of claim 24, wherein said ligand is IL-13, or a fragment of IL-13, which fragment of IL-13 retains the ability to bind to an IL-13Rα2 chain (SEQ ID NO:1).

27. A method of claim 24, wherein said ligand is a cpIL-13, which cpIL-13 retains the ability to bind to an IL-13Rα2 chain (SEQ ID NO:1).

28. A method of claim 24, wherein said ligand is a mutated IL-13, which mutated IL-13 retains the ability to bind to an IL-13Rα2 chain (SEQ ID NO:1).

29. The method of claim 24, wherein said targeting moiety is an anti-IL-13Rα2 chain antibody.

30. The method of claim 29, wherein said anti-IL-13Rα2 chain antibody is a single chain Fv or a disulfide-stabilized Fv.

31. The method of claim 24, wherein said cancer cell is a cell from a cancer selected from the group consisting of: a brain cancer, a head and neck cancer, a breast cancer, a liver cancer, a lung cancer, a mesothelioma, a colon cancer, a gastric cancer, an ovarian cancer, a renal cancer, a bladder cancer, a prostate cancer, a pancreatic cancer, a testicular cancer, a skin cancer, a cervical cancer, a uterine cancer, and a sarcoma.

32. A method of claim 31, wherein said head and neck cancer is a squamous cell carcinoma.

32. The method of claim 24, wherein the effector moiety is selected from the group consisting of cytotoxin, a radionuclide, a radioisotope, a drug, and a liposome, wherein the liposome contains a cytotoxin, a radionuclide, or a drug.

33. The method of claim 32, wherein the effector moiety is a cytotoxin.

34. The method of claim 33, wherein the cytotoxin is selected from the group consisting of ricin A, abrin, ribotoxin, ribonuclease, saporin, calicheamycin, diphtheria toxin or a subunit thereof, Pseudomonas exotoxin, a cytotoxic portion thereof, a mutated Pseudomonas exotoxin, a cytotoxic portion thereof, and botulinum toxins A through F.

35. The method of claim 34, wherein said cytotoxin is a Pseudomonas exotoxin or cytotoxic fragment thereof, or a mutated Pseudomonas exotoxin or a cytotoxic fragment thereof.

36. The method of claim 35, wherein said Pseudomonas exotoxin is selected from the group consisting of PE35, PE38, PE38KDEL, PE40, PE4E, and PE38QQR.
